SEQUENCE LISTING (31 sequences)

SEQ ID NO: 1 - DNA, 2491 bp, synthetic construct
  misc_feature 1..2491: note = synthetic DNA
  source 1..2491: mol_type = other DNA
taaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcg

SEQ ID NO: 2 - DNA, 74 bp, synthetic construct
  misc_feature 1..74: note = primer
  source 1..74: mol_type = other DNA
acatggcaaaggtagcgttgccaatgatgttacagatgagatggtcagactaaactggctgacggaatttatgc

SEQ ID NO: 3 - DNA, 20 bp, synthetic construct
  misc_feature 1..20: note = primer
  source 1..20: mol_type = other DNA
cgctaacagcgcgatttgct

SEQ ID NO: 4 - DNA, 2877 bp, synthetic construct
  misc_feature 1..2877: note = synthetic DNA
  source 1..2877: mol_type = other DNA
ggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgccatgcgagacccttgcacgccctcgctcaagccttcgtcactggtcccgccaccaaacgttggtctcggccgcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagac

SEQ ID NO: 5 - DNA, 71 bp, synthetic construct
  misc_feature 1..71: note = primer
  source 1..71: mol_type = other DNA
gcgtggagcatctggtcgcattgggtcaccagcaaatcgcgctgttagcgggcccattaagttctgtctcg

SEQ ID NO: 6 - DNA, 24 bp, synthetic construct
  misc_feature 1..24: note = primer
  source 1..24: mol_type = other DNA
gtctgaccatctcatctgtaacat

SEQ ID NO: 7 - DNA, 20 bp, synthetic construct
  misc_feature 1..20: note = primer
  source 1..20: mol_type = other DNA
catcggtgatgtcggcgata

SEQ ID NO: 8 - DNA, 67 bp, synthetic construct
  misc_feature 1..67: note = primer
  source 1..67: mol_type = other DNA
tacggggtctgacgctcagtggaacgaaaactcacgttaagggattttggcgcggaacccctatttg

SEQ ID NO: 9 - DNA, 26 bp, synthetic construct
  misc_feature 1..26: note = primer
  source 1..26: mol_type = other DNA
cactagtgaatcggccaacgcgcggg

SEQ ID NO: 10 - DNA, 25 bp, synthetic construct
  misc_feature 1..25: note = primer
  source 1..25: mol_type = other DNA
gctgagcaataactagcataacccc

SEQ ID NO: 11 - DNA, 37 bp, synthetic construct
  misc_feature 1..37: note = primer
  source 1..37: mol_type = other DNA
ggtatatctccttcttaaagttaaacaaaattatttc

SEQ ID NO: 12 - DNA, 774 bp, synthetic construct
  misc_feature 1..774: note = synthetic DNA
  source 1..774: mol_type = other DNA
atgtctatcatcgttatctctggctgtgcgacgggtattggcgcagctactcgtaaagtcctggaagcagcgggccaccagatcgttggcattgacattcgtgacgccgaggttatcgctgacctgtctaccgcagagggccgtaaacaggcgattgctgatgttctggctaagtgttctaaaggcatggatggtctggttctgtgtgcgggtctgggtccgcagaccaaagttctgggtaacgtagtgagcgttaactacttcggcgcaaccgaactgatggatgctttcctgcctgcactgaaaaaaggccatcaaccggccgcggtagtgattagcagcgttgcttctgcgcacctggcgttcgataaaaacccactggcgctggcactggaagctggcgaagaagcaaaagcccgtgcaattgtagaacacgctggtgaacagggtggtaacctggcgtacgctggctctaagaatgctctgaccgttgctgttcgtaaacgtgctgctgcctggggtgaagccggtgttcgtctgaacactatcgcgccgggtgctactgaaacgccactgctgcaagcgggcctgcaggatccacgttacggcgaatccattgctaaattcgttcctccgatgggccgtcgtgctgaaccatctgaaatggctagcgttatcgcattcctgatgtctccggctgcatcttatgttcacggtgcccagatcgtcatcgatggtggcatcgatgcagtcatgcgtcctactcaattctga

SEQ ID NO: 13 - DNA, 74 bp, synthetic construct
  misc_feature 1..74: note = primer
  source 1..74: mol_type = other DNA
caattcccctctagaaataattttgtttaactttaagaaggagatataccatgtctatcatcgttatctctggc

SEQ ID NO: 14 - DNA, 75 bp, synthetic construct
  misc_feature 1..75: note = primer
  source 1..75: mol_type = other DNA
ctcaagacccgtttagaggccccaaggggttatgctagttattgctcagctcagaattgagtaggacgcatgact

SEQ ID NO: 15 - DNA, 20 bp, synthetic construct
  misc_feature 1..20: note = primer
  source 1..20: mol_type = other DNA
cccattcgccaatccggata

SEQ ID NO: 16 - DNA, 6593 bp, synthetic construct
  misc_feature 1..6593: note = plasmid
  source 1..6593: mol_type = other DNA
taatacgactcactataggggaattgtgagcggataacaattcccctctagaaaaaagcttataattatccttacatttcccgtgagtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttaaatgtcgatagaggaaagtgtctgaaacctctagtacaaagaaaggtaagttacgtcacgggacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgttacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgatcgagcaggacggtttacatgcgggctcgcctgctgcctgggttgaacgcttatttggttacgattgggcgcagcaaaccattgggtgttcagacgcggcggtctttcgtttgtcggctcaaggtcgtcctgtgctgttcgttaaaacagatttaagcggggcgttgaacgagttgcaagatgaagcggcacgtctcagctggcttgcgactacaggagtaccgtgtgccgccgtactggatgtcgtaaccgaggccgggcgtgattggttgttgttaggtgaggtacccggacaagacctgttaagctcccatttggccccggcggaaaaggttagcattatggcggacgccatgcgtcgcttgcacaccctggaccccgcaacgtgtccgtttgatcatcaggcaaagcaccgtattgaacgtgcgcgcacacgtatggaagcggggctggtagaccaagacgacctcgatgaggaacaccaaggcctggccccggcagagttatttgcgcgcttgaaagcccgtatgcctgatggtgaagacctggtggtcacacacggggacgcatgtcttccaaacattatggtcgagaacggtcgtttctcgggctttattgattgcggacgccttggcgtcgccgatcgttaccaagatatcgcccttgcaacgcgcgacatcgcggaagaactgggtggtgagtgggcagatcgctttctggtactgtatgggattgcggccccggactcccaacgtattgctttctaccgtctgctcgatgaattcttctaataaacttgcacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcaccatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgaatgtcgagcacccgttctcggagcactgtccgaccgctttggccgccgcccagtcctgctcgcttcgctacttggagccactatcgactacgcgatcatggcgaccacacccgtcctgtggatcgccaagccgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtaaactagcctcgtttttgcatcaggaaagcagaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagtctgacgctctattcaacaaagccgccgtccatgggtagggggcttcaaatcgtccccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattgatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 17 - DNA, 364 bp, synthetic construct
  misc_feature 1..364: note = synthetic DNA
  source 1..364: mol_type = other DNA
ttcccctctagaaaatctagaataattatccttataggacgtcatggtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcgatttcctatcgatagaggaaagtgtctgaaacctctagtacaaagaaaggtaagttaaacatgacgacttatctgttatcaccacatttgtacaatctg

SEQ ID NO: 18 - DNA, 24 bp, synthetic construct
  misc_feature 1..24: note = primer
  source 1..24: mol_type = other DNA
aggggaattgtgagcggataacaa

SEQ ID NO: 19 - DNA, 18 bp, synthetic construct
  misc_feature 1..18: note = primer
  source 1..18: mol_type = other DNA
ccagctggcaattccggt

SEQ ID NO: 20 - DNA, 34 bp, synthetic construct
  misc_feature 1..34: note = primer
  source 1..34: mol_type = other DNA
ttcccctctagaaaatctagaataattatcctta

SEQ ID NO: 21 - DNA, 34 bp, synthetic construct
  misc_feature 1..34: note = primer
  source 1..34: mol_type = other DNA
cagattgtacaaatgtggtgataacagataagtc

SEQ ID NO: 22 - DNA, 22 bp, synthetic construct
  misc_feature 1..22: note = primer
  source 1..22: mol_type = other DNA
gatttggctgccagttatttag

SEQ ID NO: 23 - DNA, 21 bp, synthetic construct
  misc_feature 1..21: note = primer
  source 1..21: mol_type = other DNA
gtcctttcctcaaggttaatg

SEQ ID NO: 24 - DNA, 795 bp, synthetic construct
  misc_feature 1..795: note = synthetic DNA
  source 1..795: mol_type = other DNA
atgaacctgcgcgaaaaatacggcgaatggggtatcattctgggcgccaccgaaggcgtgggcaaagcctttgcggaaaaaattgcaagtgaaggcatgagcgtggtgctggtgggccgccgcgaagaaaaactgcaggaactgggcaaaagcattagcgaaacctatggcgttgatcatatggtgattcgcgccgattttgcgcagagcgattgcaccgataaaatttttgaagcgaccaaagatctggatatgggctttatgagttacgtggcatgctttcatacctttggcaaactgcaggataccccgtgggaaaaacatgaacagatgattaacgtgaatgttatgacctttctgaaatgcttctatcattatatgggcatttttgccaaacaggatcgcggcgcggtaattaatgtgagcagcctgaccgcgattagtagcagcccgtataacgcgcagtatggcgcgggcaaatcgtacattaaaaaactgacggaagccgtggcggccgaatgcgaaagcaccaatgtggacgtggaagtcattaccctgggcaccgtgattaccccgagcctgctgagcaacctgccgggcggcccggccggcgaagccatgatgaaaaccgcgatgacgccggaagcctgcgtggaagaagcgtttgacaacctgggcaaaagcctgagcgtgattgcgggcgaacacaacaaagccaatgttcataactggcaggcgaacaaaaccgatgatgaatatattcgctatatgggcagcttttatagcaataactaa

SEQ ID NO: 25 - DNA, 72 bp, synthetic construct
  misc_feature 1..72: note = primer
  source 1..72: mol_type = other DNA
caattcccctctagaaataattttgtttaactttaagaaggagatataccatgaacctgcgcgaaaaatacg

SEQ ID NO: 26 - DNA, 75 bp, synthetic construct
  misc_feature 1..75: note = primer
  source 1..75: mol_type = other DNA
ctcaagacccgtttagaggccccaaggggttatgctagttattgctcagcttagttattgctataaaagctgccc

SEQ ID NO: 27 - DNA, 23 bp, synthetic construct
  misc_feature 1..23: note = primer
  source 1..23: mol_type = other DNA
ccaaaatcccttaacgtgagttt

SEQ ID NO: 28 - DNA, 30 bp, synthetic construct
  misc_feature 1..30: note = primer
  source 1..30: mol_type = other DNA
gaattaattcatgagcggatacatatttga

SEQ ID NO: 29 - DNA, 966 bp, synthetic construct
  misc_feature 1..966: note = synthetic DNA
  source 1..966: mol_type = other DNA
cgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 30 - DNA, 75 bp, synthetic construct
  misc_feature 1..75: note = primer
  source 1..75: mol_type = other DNA
tacggggtctgacgctcagtggaacgaaaactcacgttaagggattttggcgcggaacccctatttgtttatttt

SEQ ID NO: 31 - DNA, 75 bp, synthetic construct
  misc_feature 1..75: note = primer
  source 1..75: mol_type = other DNA
gtttatttttctaaatacattcaaatatgtatccgctcatgaattaattcttaccaatgcttaatcagtgaggca

(19) United States
(12) Patent Application Publication
(10) Pub. No.: US 2025/0296949 A1
(43) Pub. Date: Sep. 25, 2025

(54) HIGH PURITY NON-ANIMAL DERIVED UDCA

(71) Applicant: SANDHILL ONE, LLC, West Palm Beach, FL (US)

(72) Inventors: J Gregory REID, West Palm Beach, FL (US); Daniel John GANLEY, West Palm Beach, FL (US); Jayachandra P REDDY, West Palm Beach, FL (US)

(73) Assignee: SANDHILL ONE, LLC, West Palm Beach, FL (US)

(21) Appl. No.: 18/704,848

(22) PCT Filed: Nov. 1, 2022
(86) PCT No.: PCT/US2022/079080

§ 371 Date: Apr. 25, 2024

Related U.S. Application Data
(60) Provisional application No. 63/274,532, filed on Nov. 2, 2021.

(51) Int. Cl. C07J 9/00

(52) U.S. Cl. CPC C07J 9/005

(57) ABSTRACT

Methods of making cholic acid derivatives, particularly UDCA, from non-animal sources, having exceptional purity and therapeutic utility.

### RELATED APPLICATIONS

This application claims priority to U.S. Ser. No. 63/274,532, filed Nov. 2, 2021, the content of which is incorporated by reference as if fully set forth herein.

### SEQUENCE LISTING INCORPORATION BY REFERENCE

The application herein incorporates by reference in its entirety the sequence listing material in the ASCII text file named “sequence listing”, created Nov. 11, 2024, and having the size of 57 kilobytes, filed with this application.

### FIELD OF THE INVENTION

The present invention relates generally to cholic acid derivatives, particularly UDCA, having exceptional purity and therapeutic utility, preferably derived from non-animal sources, and to methods and intermediates for making same.

### BACKGROUND OF THE INVENTION

Cholic acid and its derivatives find utility in numerous medical applications and research initiatives. Cholic acid itself, sold under the brand name Cholbam®, is approved for use as a treatment for children and adults with bile acid synthesis disorders due to single enzyme defects, and for peroxisomal disorders (such as Zellweger syndrome). 7-Ketolithocholic acid has been examined for its effect on endogenous bile acid synthesis, biliary cholesterol saturation, and its possible role as a precursor of chenodeoxycholic acid and ursodeoxycholic acid. See Salen et al. Gasteroenterology, 1982; 83:341-7. Ursodeoxycholic acid (a/k/a UDCA or ursodiol), sold under the brand name URSO 250® and URSO Forte® tablets, is approved for the treatment of patients with primary biliary cirrhosis (PBC). More recently, obeticholic acid, sold under the brand name Ocaliva®, was approved for the treatment of PBC in combination with UDCA in adults with an inadequate response to UDCA, or as monotherapy in adults unable to tolerate UDCA.

In spite of this significant medical interest in cholic acid derivatives, methods of synthesizing the derivatives remain a cumbersome inefficient process, with numerous processes being proposed. Fantin et al. Steroids, 1993 November; 58:524-526, discloses the preparation of 7α-, 12α-, 12β-hydroxy and 7α-, 12α- and 7α-, 12β-dihydroxy-3-ketocholanoic acids by protecting the 3-keto group as dimethyl ketal and subsequent reduction with sodium borohydride of the corresponding 7- and 12-oxo functionalities. WO 2017/079062 A1 by Galvin reports a method of preparing obeticholic acid by direct alkylation at the C-6 position of 7-keto lithocholic acid (KLCA). He et al., Steroids, 2018 December; 140:173-178, discloses a synthetic route of producing ursodeoxycholic acid (UDCA) and obeticholic acid (OCA) through multiple reactions from cheap and readily-available cholic acid. Wang et al., Steroids 157 (2020) 108600, similarly report a synthetic route of producing ursodeoxycholic acid (UDCA) through multiple reactions from commercially available bisnoralcohol (BA). The process is not stereospecific at the three involved chiral centers, requires chromatographic purification, and still produces a product contaminated by chiral impurities.

Commercially available preparations containing bile acids such as UDCA are derived exclusively from animal corpses such as cows and sheep, which pose the threat of contamination by pathogens such as prions and other toxins. In addition, even though bile acids from animal sources are typically purified in order to exclude impurities, in practice, such purified compositions contain a mixture of bile acids due to the difficulty separating closely related analogs and isomers. The United States Pharmacopoeia explicitly permits CDCA in UDCA, and Rajevic (1998) report that all commercially available compositions of UDCA of animal origin that he tested contained some chenodeoxycholic acid (CDCA). Rajevic M and Betto P, J. Liq. Chrom. & Rel. Technol., 21(18), 2821-2830 (1998).

What is needed are more efficient processes for making cholic acid derivatives, especially UDCA. A particular need exists for the production of non-animal derived cholic acid derivatives, and processes that eliminate the production of harmful analogs, isomers, and contaminants associated with UDCA.

### SUMMARY OF INVENTION

The inventors have for the first time developed non-animal derived UDCA which differs from UDCA in the prior art, particularly non-animal-derived UDCA in the prior art, by the substantial absence of several prominent impurities, including 3β-hydroxysteroids, 5α-steroids, and 7α-hydroxysteroids, especially CDCA. The UDCA can be distinguished from animal derived UDCA by its δ13C signature. Thus, in a first principal embodiment the invention provides a compound selected from ursodeoxycholic acid of formula I:

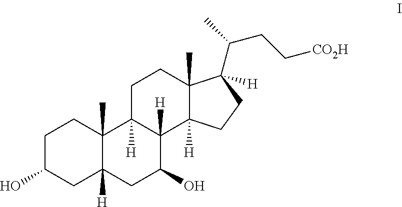

and its pharmaceutically acceptable salts comprising a δ13C value corresponding to a plant derived molecule, preferably comprising less than −15‰, −17.5‰, −20‰, −22.5‰, or −25‰ δ¹³C relative to VPDB, and an impurity profile characterized by: (a) less than 5%, 3%, 1%, 0.5%, 0.1%, 0.05%, 0.03%, or 0.01% of any 3β-hydroxysteroids; (b) less than 5%, 3%, 1%, 0.5%, 0.1%, 0.05%, 0.03%, or 0.01% of any 5α-steroids; and/or (c) less than 5%, 3%, 1%, 0.5%, 0.1%, 0.05%, 0.03%, or 0.01% of any 7α-hydroxysteroids.

In a second principal embodiment the invention provides a compound selected from ursodeoxycholic acid of formula I:

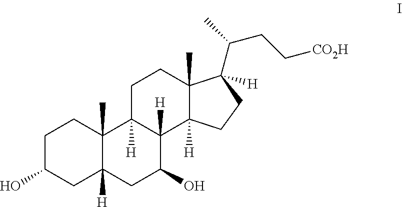

and its pharmaceutically acceptable salts comprising an impurity profile characterized by (a) less than 0.05%, 0.03%, or 0.01% of any 3β-hydroxysteroids, and/or (b) less than 0.05%, 0.03%, or 0.01% of any 5α-steroids, and/or (c) less than 0.05%, 0.03%, or 0.01% of any 7α-steroids.

Methods have been developed for producing UDCA that substantially reduce or eliminate the production of 3β- and 7α-hydroxysteroids from the manufacturing process. Thus, in a third principal embodiment the invention provides a method of producing the compound of the first or second principal embodiment that goes through a DKCA intermediate, comprising: (a) contacting the DKCA with a 3α-hydroxysteroid dehydrogenase to stereo-selectively reduce the DKCA to a 3a hydroxy intermediate, and contacting the 3a hydroxy intermediate with a 7β-hydroxysteroid dehydrogenase to stereo-selectively reduce the 3a hydroxy intermediate to UDCA; (b) contacting the DKCA with a 7β-hydroxysteroid dehydrogenase to stereo-selectively reduce the DKCA to a 7β hydroxy intermediate, and contacting the 7β hydroxy intermediate with a 3α-hydroxysteroid dehydrogenase to stereo-selectively reduce the 7β hydroxy intermediate to UDCA; or (c) simultaneously contacting the DKCA with a 3α-hydroxysteroid dehydrogenase and a 7β-hydroxysteroid dehydrogenase to stereo-selectively reduce the DKCA to UDCA. The DKCA is optionally provided as or derived from an ethylenediamine salt of DKCA (optionally Pattern 6-D), a tert-butylamine salt of DKCA (optionally Pattern 9-A), or a diisopropylamine salt of DKCA (optionally Pattern 10-A). In a preferred embodiment, the DKCA is first provided in an isolated state.

Methods also have been developed for producing UDCA that substantially reduces or eliminates the presence of 5α-steroids in the final product. Thus, in a fourth principal embodiment the invention provides a method of producing the compound of the first or second principal embodiment, made by a process that goes through a 4,5 unsaturated 3,7-diketo DKCA precursor, comprising contacting a 4,5 unsaturated 3,7-diketo DKCA precursor with a Pd catalyst in the presence of pyridine or a pyridine derivative, thereby hydrogenating the 4,5 double bond to produce DKCA.

In a fifth principal embodiment the invention provides an ethylenediamine salt of 3,7-DKCA, preferably in crystalline form characterized by Pattern 6-D.

In a sixth principal embodiment the invention provides a tert-butylamine salt of 3,7-DKCA, preferably in crystalline form characterized by Pattern 9-A.

In a seventh principal embodiment the invention provides a diisopropylamine salt of 3,7-DKCA, preferably in crystalline form characterized by Pattern 10-A.

Additional advantages of the invention are set forth in part in the description that follows, and in part will be obvious from the description, or may be learned by practice of the invention.

The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 is an HPLC chromatogram of the 3,7-DKCA starting material used in Example 10. The peak at 5.482 min is the 5-α stereoisomer.

FIG. 2 is an HPLC chromatogram of 3,7-DKCA produced by the method of Example 10. The 5-α stereoisomeric impurity is not detected.

FIG. 3 is a high-resolution XRPD diffractogram of scaled-up Pattern 9-A from salt formation of 3,7-DKCA with tert-butylamine in ethanol.

FIG. 4 is a high-resolution XRPD diffractogram of scaled-up Pattern 6-D from salt screening of 3,7-DKCA with ethylenediamine in IPA:water (9:1 vol.).

FIG. 5 is a high-resolution XRPD diffractogram of scaled-up Pattern 10-A from salt screening of 3,7-DKCA with diisopropylamine in MIBK/heptane.

FIG. 6 is an HPLC chromatogram of tert-butylamine salt of 3,7-DKCA produced substantially according to the 3-picoline solvent hydrogenation and tert-butylamine crystallization methods described herein.

### DETAILED DESCRIPTION OF THE INVENTION

### Definitions and Use of Terms

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As used in the specification and claims, the singular forms a, an, and the include plural references unless the context clearly dictates otherwise. For example, the term “a specification” refers to one or more specifications for use in the presently disclosed methods and systems. “A hydrocarbon” includes mixtures of two or more such hydrocarbons, and the like.

When the term “any” is used herein, in reference to the lack of contaminants or impurities, it will be understood that the term includes zero % but that some contaminants or impurities can also be present, but always below the limit of detection (typically <0.05% or <0.03%).

The word “or” or like terms as used herein means any one member of a particular list and also includes any combination of members of that list. Thus, when a list comprises “A, B, or C,” the list could alternatively be written as comprising “A, B, C, or a combination thereof,” or as comprising “A, B, C, A+B, A+C, B+C, or A+B+C.”

As used in this specification and in the claims which follow, the word “comprise” and variations of the word, such as “comprising” and “comprises,” means “including but not limited to,” and is not intended to exclude, for example, other additives, components, integers or steps. When an element is described as comprising one or a plurality of components, steps or conditions, it will be understood that the element can also be described as “consisting of” or “consisting essentially of” the component, step or condition, or the plurality of components, steps or conditions.

When ranges are expressed herein by specifying alternative upper and lower limits of the range, it will be understood that the endpoints can be combined in any manner that is mathematically feasible. Thus, for example, a range of from 50 or 80 to 100 or 70 can alternatively be expressed as a series of ranges of from 50 to 100, from 50 to 70, and from 80 to 100. When a series of upper bounds and lower bounds are related using the phase and/or, it will be understood that the upper bounds can be unlimited by the lower bonds or combined with the lower bounds, and vice versa. Thus, for example, a range of greater than 40% and/or less than 80% includes ranges of greater than 40%, less than 80%, and greater than 40% but less than 80%.

When used herein the term “about” will compensate for variability allowed for in the pharmaceutical industry and inherent in pharmaceutical products. In one embodiment the term allows for any variation within 5% of the recited specification or standard. In one embodiment the term allows for any variation within 10% of the recited specification or standard.

Ursodeoxycholic acid, 3α,7β-dihydroxy-5β-cholanic acid, or simply ursodiol or UDCA, is an epimer of chenodeoxycholic acid having the following chemical structure:

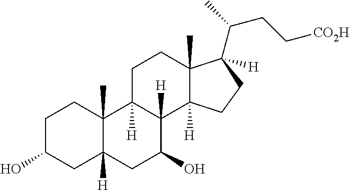

UDCA can exist as a free acid or a salt. When expressed without specifying the free acid or salt form, the term “UDCA” or “ursodeoxycholic acid” will be understood to encompass both the free acid and its salts. Using the methods of the current invention, UDCA can be derived from plant and animal sources, and combinations of plant and animal sources. When UDCA is expressed without specifying its source, it will be understood to encompass UDCA from any source, and with any δ¹³C content.

DKCA, or 3,7-DKCA, or 3,7-diketo-50-cholanic acid, is represented by the following chemical structure: —

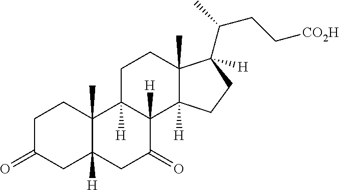

CDCA, or chenodeoxycholic acid, is represented by the following chemical structure.

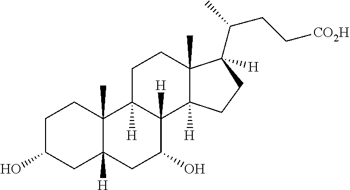

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use or use in a dietary supplement. “Pharmaceutically acceptable salts” means salts that are pharmaceutically acceptable, as defined above, and which possess the desired pharmacological or chemical activity.

“Fossil carbon percentage” means the percentage of carbon atoms in a molecule derived from “synthetic” (petrochemical) sources. “Fossil/animal” means derived exclusively from fossil sources, derived exclusively from animal sources, or derived from fossil and animal sources.

“δ¹³C value” is an isotopic measurement of the delta notation of ¹³C. δ¹³C values are expressed as a per mil (‰) deviation, e.g. per one thousand, from an internationally accepted PDB standard (originally a carbonate from the Pee Dee Belemnite formation in South Carolina but more commonly today Vienna Pee Dee Belemnite (VPDB)). δ¹³C values are determined using the following formula:

$${δ}^{13} C = ( {( {δ}^{13} C / {δ}^{12} C )}_{sample} - ( {( {δ}^{13} C / {δ}^{12} C )}_{PDB} ) / ( {( {δ}^{13} C / {δ}^{12} C )}_{PDB} ) * 1000$$

By “plant sources” are meant any source, which may be defined as a plant such as for example trees, shrubs, herbs, grasses, ferns, mosses, flowers, vegetables, and weeds, as well as compounds derived from plants such as phytosterols, and phytosterol derivatives. The plant can be a C3 plant, a C4 plant, or a combination of both.

The term “plant derived” refers to a molecule comprising a δ¹³C value corresponding to a plant derived molecule or a mixed fossil/animal and plant derived molecule, comprising a majority of plant-derived carbons. A plant derived molecule can thus be characterized as having greater than 50%, 75%, 90%, 95%, 98%, or 99% plant derived carbons, with the remaining carbons (if any) derived from fossil/animal resources.

By “C3 plants” are meant plants that do not have photosynthetic adaptations to reduce photorespiration. This includes plants such as rice, wheat, soybeans, most fruits, most vegetables and all trees.

By “C4 plants” are meant plants where the light-dependent reactions and the Calvin cycle are physically separated and where the light-dependent reactions occur in the mesophyll cells and the Calvin cycle occurs in bundle-sheath cells. This includes plants such as crabgrass, sugarcane, sorghum and corn.

### DISCUSSION OF PRINCIPAL EMBODIMENTS

The invention can be defined based on several principal embodiments which can be combined in any manner physically and mathematically possible to create additional principal embodiments.

Thus, in a first principal embodiment the invention provides a compound selected from ursodeoxycholic acid of formula I:

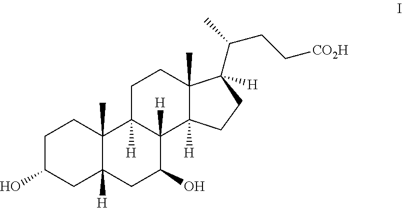

and its pharmaceutically acceptable salts comprising a δ¹³C value corresponding to a plant derived molecule, preferably comprising less than −15‰, −17.5‰, −20‰, −22.5‰, or −25‰ δ¹³C relative to VPDB, and an impurity profile characterized by: (a) less than 5%, 3%, 1%, 0.5%, 0.1%, 0.05%, 0.03%, or 0.01% of any 3β-hydroxysteroids; (b) less than 5%, 3%, 1%, 0.5%, 0.1%, 0.05%, 0.03%, or 0.01% of any 5α-steroids; and/or (c) less than 5%, 3%, 1%, 0.5%, 0.1%, 0.05%, 0.03%, or 0.01% of any 7α-hydroxysteroids.

In a second principal embodiment the invention provides a compound selected from ursodeoxycholic acid of formula I:

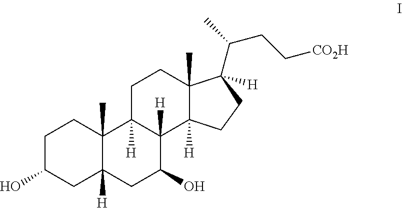

and its pharmaceutically acceptable salts comprising an impurity profile characterized by (a) less than 0.05%, 0.03%, or 0.01% of any 3β-hydroxysteroids, and/or (b) less than 0.05%, 0.03%, or 0.01% of any 5α-steroids, and/or (c) less than 0.05%, 0.03%, or 0.01% of any 7α-steroids.

In a third principal embodiment the invention provides a method of producing the compound of the first or second principal embodiment that goes through a DKCA intermediate, comprising: (a) contacting the DKCA with a 3α-hydroxysteroid dehydrogenase to stereo-selectively reduce the DKCA to a 3α hydroxy intermediate, and contacting the 3α hydroxy intermediate with a 7β-hydroxysteroid dehydrogenase to stereo-selectively reduce the 3α hydroxy intermediate to UDCA; (b) contacting the DKCA with a 7β-hydroxysteroid dehydrogenase to stereo-selectively reduce the DKCA to a 7β hydroxy intermediate, and contacting the 7β hydroxy intermediate with a 3α-hydroxysteroid dehydrogenase to stereo-selectively reduce the 7β hydroxy intermediate to UDCA; or (c) simultaneously contacting the DKCA with a 3α-hydroxysteroid dehydrogenase and a 7β-hydroxysteroid dehydrogenase to stereo-selectively reduce the DKCA to UDCA. Preferred sources for the DKCA include the ethylenediamine salt of 3,7-DKCA (preferably crystalline form 6-D), the tert-butylamine salt of 3,7-DKCA (preferably crystalline form 9-A), and the diisopropylamine salt of 3,7-DKCA (preferably crystalline form 10-A). In a preferred embodiment, the 3,7-DKCA is provided in an isolated state.

In a fourth principal embodiment the invention provides a method of producing the compound of the first or second principal embodiment, made by a process that goes through a 4,5 unsaturated 3,7-diketo DKCA precursor, comprising contacting a 4,5 unsaturated 3,7-diketo DKCA precursor with a Pd catalyst in the presence of pyridine or a pyridine derivative, thereby hydrogenating the 4,5 double bond to produce DKCA.

In a fifth principal embodiment the invention provides an ethylenediamine salt of 3,7-DKCA (preferably crystalline form 6-D).

In a sixth principal embodiment the invention provides a tert-butylamine salt of 3,7-DKCA (preferably crystalline form 9-A).

In a seventh principal embodiment the invention provides a diisopropylamine salt of 3,7-DKCA (preferably crystalline form 10-A).

### Discussion of Subembodiments

The invention can further be understood with reference to various subembodiments which can modify any of the principal embodiments. These subembodiments can be combined in any manner that is both mathematically and physically possible to create additional subembodiments, which in turn can modify any of the principal embodiments. For example, any of the subembodiments requiring a plant-derived UDCA can be used to further modify the UDCA embodiments not limited by plant origin. In like manner, any of the purity subembodiments can be used to further modify an embodiment with broader purity allowances.

In any of the purity embodiments or subembodiments of the current invention, it will be understood that some measure of impurity can also be present (even if non-detectable by current analytical techniques), or that none can be present, and that when the impurity is present, it is preferably present in an amount greater than 0.001% or 0.005%. Thus:
whenever a compound is stated to contain less than a certain percentage of 3β-hydroxysteroids or 3β-UDCA, it will be understood that the compound can also be expressed in alternative embodiments as containing greater than 0.001% or 0.005% 3β-hydroxysteroids or 3β-UDCA;
whenever a compound is stated to contain less than a certain percentage of 5α-steroids or 5α-UDCA, it will be understood that the compound can also be expressed in alternative embodiments as containing greater than 0.001% or 0.005% 5α-steroids or 5α-UDCA;
whenever a compound is stated to contain less than a certain percentage of 7α-hydroxysteroids or CDCA, it will be understood that the compound can also be expressed in alternative embodiments as containing greater than 0.001% or 0.005% 7α-hydroxysteroids or CDCA;

Similarly, when UDCA is referred to herein as plant derived, it will be understood that the UDCA will preferably comprise less than −15‰, −170.5‰, −20‰, −22.5‰, or −25‰ δ¹³C relative to VPDB, most preferably less than −20‰ δ¹³C relative to VPDB.

In one embodiment, the invention provides plant derived UDCA comprising less than 5%, 3%, 1%, 0.5%, 0.1%, 0.05%, 0.03%, or 0.01% of any 3β-hydroxysteroids.

In another embodiment, the invention provides plant derived UDCA comprising less than 5%, 3%, 1%, 0.5%, 0.1%, 0.05%, 0.03%, or 0.01% of any 5α-steroids.

In another embodiment, the invention provides plant derived UDCA comprising less than 5%, 3%, 1%, 0.5%, 0.1%, 0.05%, 0.03%, or 0.01% of any 7α-hydroxysteroids.

In another embodiment, the invention provides plant derived UDCA comprising: (a) less than 5%, 3%, 1%, 0.5%, 0.1%, 0.05%, 0.03%, or 0.01% of any 3β-hydroxysteroids; (b) less than 5%, 3%, 1%, 0.5%, 0.1%, 0.05%, 0.03%, or 0.01% of any 5α-steroids; and (c) less than 5%, 3%, 1%, 0.5%, 0.1%, 0.05%, 0.03%, or 0.01% of any 7α-hydroxysteroids.

In another embodiment, the invention provides plant derived UDCA comprising: (a) less than 1% of any 3β-hydroxysteroids; (b) less than 1% of any 5α-steroids; and (c) less than 1% of any 7α-hydroxysteroids.

In another embodiment, the invention provides plant derived UDCA comprising: (a) less than 0.1% of any 3β-hydroxysteroids; (b) less than 0.5% of any 5α-steroids; and (c) less than 0.1% of any 7α-hydroxysteroids.

In another embodiment, the invention comprises UDCA comprising an impurity profile characterized by less than 0.05%, 0.03%, or 0.01% of any 3β-hydroxysteroids.

In another embodiment, the invention comprises UDCA comprising an impurity profile characterized by less than 0.05%, 0.03%, or 0.01% of any 3β-hydroxysteroids, and less than 1% or 0.5% of impurities selected from starting materials, by-products, intermediates, and degradation products.

In another embodiment, the invention comprises UDCA in the absence of any 3β-hydroxysteroids.

In another embodiment, the invention comprises UDCA in the absence of any 3β-hydroxysteroids and less than 1% or 0.5% of impurities selected from starting materials, by-products, intermediates, and degradation products.

In another embodiment, the invention comprises UDCA comprising an impurity profile characterized by less than 0.05%, 0.03%, or 0.01% of any 5α-steroids.

In another embodiment, the invention comprises UDCA comprising an impurity profile characterized by less than 0.05%, 0.03%, or 0.01% of any 5α-steroids, and less than 1% or 0.5% of impurities selected from starting materials, by-products, intermediates, and degradation products.

In another embodiment, the invention comprises UDCA in the absence of any 5α-steroids.

In another embodiment, the invention comprises UDCA in the absence of any 5α-steroids and less than 1% or 0.5% of impurities selected from starting materials, by-products, intermediates, and degradation products.

In another embodiment, the invention comprises UDCA comprising an impurity profile characterized by less than 0.05%, 0.03%, or 0.01% of any 7α-hydroxysteroids.

In another embodiment, the invention comprises UDCA comprising an impurity profile characterized by less than 0.05%, 0.03%, or 0.01% of any 7α-hydroxysteroids, and less than 1% or 0.5% of impurities selected from starting materials, by-products, intermediates, and degradation products.

In another embodiment, the invention comprises UDCA in the absence of any 7α-hydroxysteroids.

In another embodiment, the invention comprises UDCA in the absence of any 7α-hydroxysteroids and less than 1% or 0.5% of impurities selected from starting materials, by-products, intermediates, and degradation products.

The UDCA of the current invention can further comprise: (i) less than 5%, 3%, 1%, 0.5%, 0.1%, 0.05%, 0.03%, or 0.01% of LCA; (ii) less than 5%, 3%, 1%, 0.5%, 0.1%, 0.05%, 0.03%, or 0.01% of any 3-keto, 7-hydroxysteroids; (iii) less than 5%, 3%, 1%, 0.5%, 0.1%, 0.05%, 0.03%, or 0.01% of any 3-hydroxy, 7-ketosteroids; (iv) less than 5%, 3%, 1%, 0.5%, 0.1%, 0.05%, 0.03%, or 0.01% of DKCA, and/or (v) a combination thereof.

Particularly preferred UDCA of the current invention is free from any 7α-hydroxysteroids.

The UDCA of the current invention preferably comprises less than 3%, 2%, or 1% of impurities selected from starting materials, by-products, intermediates, and degradation products.

In like manner, the UDCA of the current invention preferably is present in an isolated state.

The inventive compounds derive particularly from the ability to control/eliminate the production of 3β-hydroxysteroids and 7α-hydroxysteroids using the ketoreductases of the present invention. Therefore, in still further embodiments the invention provides UDCA having an impurity profile characterized by less than 0.5%, 0.1%, 0.05%, 0.03%, or 0.01% of any 3β-hydroxysteroids and less than 0.5%, 0.1%, 0.05%, 0.03%, or 0.01% of any 7α-hydroxysteroids, made by a process that goes through a DKCA intermediate, comprising: (a) contacting the DKCA with a 3α-hydroxysteroid dehydrogenase to stereo-selectively reduce the DKCA to a 3α hydroxy intermediate, and contacting the 3α hydroxy intermediate with a 7β-hydroxysteroid dehydrogenase to stereo-selectively reduce the 3α hydroxy intermediate to UDCA; (b) contacting the DKCA with a 7β-hydroxysteroid dehydrogenase to stereo-selectively reduce the DKCA to a 7β hydroxy intermediate, and contacting the 7β hydroxy intermediate with a 3α-hydroxysteroid dehydrogenase to stereo-selectively reduce the 7β hydroxy intermediate to UDCA; or (c) simultaneously contacting the DKCA with a 3α-hydroxysteroid dehydrogenase and a 7β-hydroxysteroid dehydrogenase to stereo-selectively reduce the DKCA to UDCA.

The 3,7-DKCA is preferably provided as or derived from an ethylenediamine salt of 3,7-DKCA (optionally Pattern 6-D), a tert-butylamine salt of 3,7-DKCA (optionally Pattern 9-A), or a diisopropylamine salt of 3,7-DKCA (optionally Pattern 10-A). When going through a salt it will be understood that the salt can first be removed from the 3,7-DKCA before ketoreduction. It will also be understood that the 3,7-DKCA can be isolated before undergoing the ketoreductase conversion.

The inventive compounds also derive from the novel 3,7-DKCA crystalline salts disclosed herein.

In one embodiment the DKCA is derived from the ethylenediamine salt of 3,7-DKCA, preferably a crystalline form defined by Pattern 6-D.

In another embodiment the DKCA is derived from the tert-butylamine salt of 3,7-DKCA, preferably a crystalline form defined by Pattern 9-A.

In still another embodiment, the DKCA is derived from the diisopropylamine salt of 3,7-DKCA, preferably a crystalline form defined by Pattern 10-A.

The inventive compounds also derive from the ability to control the production of 5α-steroids, particularly early in the synthetic process, which results in practically eliminating the 5α-steroids from the final product. Therefore, in additional embodiments the invention provides UDCA comprising an impurity profile characterized by less than 1.0%, 0.5%, 0.1%, 0.05%, 0.03%, or 0.01% of any 5α-steroids, made by a process that goes through a 4,5 unsaturated 3,7-diketo DKCA, comprising contacting the precursor with a Pd catalyst in the presence of pyridine or a pyridine derivative, thereby hydrogenating the 4,5 double bond to produce DKCA.

### Novel Salts, Hydrates, and Crystal Forms

When any compound is referenced herein, either by itself, in combination with other ingredients, or in a chemical or biological process, it will be understood that the compound can be present in or used as an isolated form. By isolated form is meant that the compound is preferably present as a solid, and that it is substantially free of any compounds other than the recited compound (i.e. <10%, 5%, 3%, or 1% other compounds).

A preferred form of the ethylenediamine salt of 3,7-DKCA is a crystalline form defined by Pattern 6-D. When reference is made to a crystalline form defined by Pattern 6-D, it will be understood that the crystalline form
has an XRPD pattern comprising at least one, two, or three peaks in terms of 20, selected from the group consisting of 5.81, 8.69, 9.95, 10.92, 11.60, 13.08, 13.78, 14.59, 16.03, 16.51, 25.11, 27.42, 28.82, 30.24, 33.35, and 38.22°±0.2°,
has an XRPD pattern comprising at least one, two, or three peaks in terms of 20, selected from the group consisting of 5.81, 9.95, 10.92, 13.08, 14.69, and 16.03°±0.2°,
has an XRPD pattern comprising at least one, two, or three peaks in terms of 20, selected from the group consisting of 17.40, 17.77, 18.11, 18.89, 19.21, 19.94, 20.27, 210.4, 21.32, 23.45, 26.00, 26.23, 28.10, 28.33, 37.52, and 37.83°±0.1°,
has an XRPD pattern comprising at least one, two, or three peaks in terms of 20, selected from the group consisting of 17.40, 17.77, 18.89, 19.21, 19.94, and 23.450±0.1°, or
has an XRPD pattern substantially as depicted in FIG. 4.

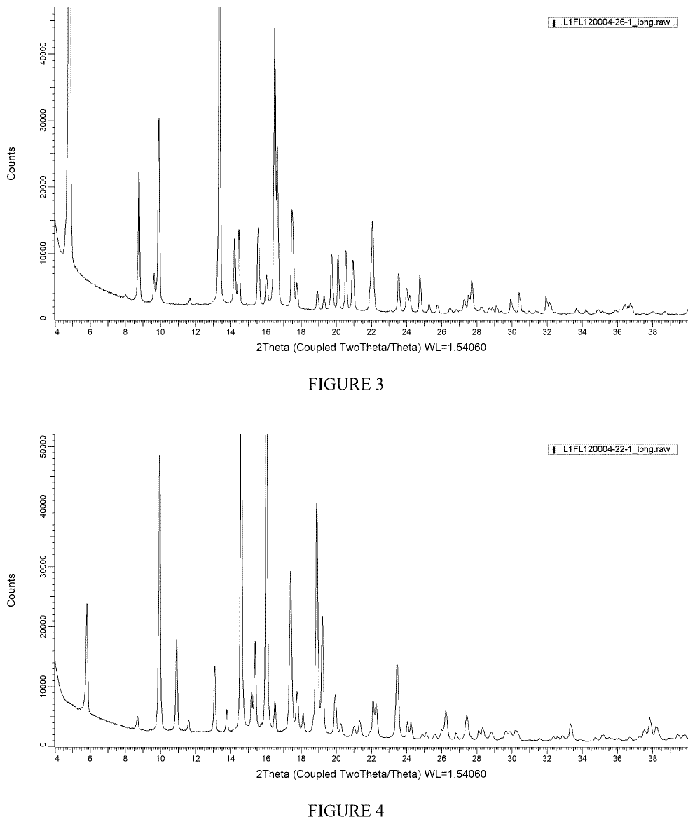

A preferred form of the tert-butylamine salt of 3,7-DKCA is a crystalline form defined by Pattern 9-A. When reference is made to a crystalline form defined by Pattern 9-A, it will be understood that the crystalline form
has an XRPD pattern comprising at least one, two, or three peaks in terms of 20, selected from the group consisting of 4.83, 8.77, 13.35 15.56, 16.03, 20.54, 22.05, 23.53, 24.75, 29.93, 30.40, and 31.97°±0.2°,
has an XRPD pattern comprising at least one, two, or three peaks in terms of 20, selected from the group consisting of 4.83, 8.77, 13.35, 15.56, and 22.05±0.2°,
has an XRPD pattern comprising at least one, two, or three peaks in terms of 20, selected from the group consisting of 9.68, 9.90, 14.22, 14.46, 17.51, 17.76, 18.92, 19.30, 19.73, 20.10, 20.95, and 27.26°±20 0.1°,
has an XRPD pattern comprising at least one, two, or three peaks in terms of 20, selected from the group consisting of 9.90, 14.22, 14.46, 17.51, 19.73, and 20.10°±0.1°, or
has an XRPD pattern substantially as depicted in FIG. 3.

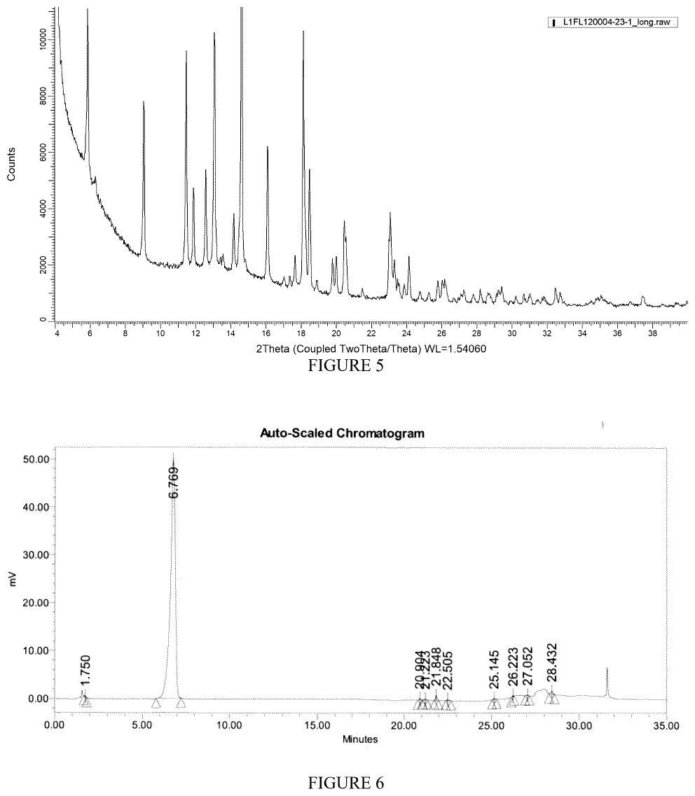

A preferred form of the diisopropylamine salt of 3,7-DKCA is a crystalline form defined by Pattern 10-A. When reference is made to a crystalline form defined by Pattern 10-A, it will be understood that the crystalline form
has an XRPD pattern comprising at least one, two, or three peaks in terms of 20, selected from the group consisting of 5.85, 6.29, 9.05, 12.58, 14.17, 16.09, 18.13, 18.47, 18.89, 20.49, 21.48, 24.75, 25.27, 28.65, 30.21, 31.82, 34.78, and 37.44°±0.2°,
has an XRPD pattern comprising at least one, two, or three peaks in terms of 20, selected from the group consisting of 5.85, 9.05, 12.58, 14.17, 16.09, 18.13, 18.47, and 20.49°±0.2°,
has an XRPD pattern comprising at least one, two, or three peaks in terms of 20, selected from the group consisting of 11.46, 11.88, 13.07, 14.61, 14.82, 17.03, 17.37, 17.65, 19.79, 20.00, 23.08, 23.86, 24.13, 25.78, 27.80, 28.19, 30.66, 31.02, 32.46, 32.74, and 35.28°±0.1°,
has an XRPD pattern comprising at least one, two, or three peaks in terms of 20, selected from the group consisting of 11.46, 11.88, 13.07, 14.61, 20.00, 23.08, and 24.13°±0.1°, or
has an XRPD pattern substantially as depicted in FIG. 5.

### Carbon Sources

The carbon source may be a steroid, such as cholesterol, stigmasterol, campesterol and sitosterol or mixtures of all of them, preferably sitosterol. Preferably, the carbon source will be a plant phytosterol such as sitosterol, stigmasterol, campesterol and brassicasterol or a mixture thereof. In one embodiment, the phytosterols are mainly of soybean or tall oil origin.

The advantages of using a plant derived source over current animal sources are numerous. There is a virtually unlimited capacity of starting materials, the quality of the product can be more easily monitored and controlled, there is no risk of spreading pathogens carried in mammalian specified, and there is no risk of significant supply chain disruptions due to animal pandemics.

The origin of the carbon atoms may be even further differentiated by measurement of the δ¹³C value as disclosed e.g. in U.S. Pat. No. 8,076,156 and “Stable Isotope Ratios as biomarkers of diet for health research” by D. M. O'Brien, Annual Reviews (www.annualreviews.org), 2015. The δ-value appears as the ¹³C is measured in relation to a standard being Pee Dee Belemnite based on a Cretaceous marine fossil, which had an anomalously high ¹³C. Biochemical reactions discriminate against ¹³C, which is why the concentration of ¹²C is increased in biological materials. In this manner, different sources such as plant versus animal may be distinguished using the pure compounds as reference values as described in Application Note 30276 from Thermo Scientific: “Detection of Squalene and Squalane Origin with Flash Elemental Analyzer and Delta V Isotope Ratio Mass Spectrometer” by Guibert et al. (2013).

Isotope ratios are conveniently quantified in parts per mil (‰) in what is called the 6 notation. Specifically, δ¹³C=(Rₛₐₘₚₗₑ/R_{standard}−1)×1,000 where Rₛₐₘₚₗₑ is the ¹³C/¹²C isotope ratio of the sample and R_{standard} is 0.0112372, which is based on the standard Vienna PeeDee Belemnite (VPDB) value. Thus, 1 unit of ¹³C represents a change of ˜1 in the fifth decimal place of the ¹³C/¹²C isotope ratio. Further discussion of the technique can be found, for example, in R. N. Zare et al., High-precision optical measurements of ¹³C/¹²C isotope ratios in organic compounds at natural abundance. 10928-10932, PNAS Jul. 7, 2009, vol. 106 no. 27.

The δ¹³C values may also differ among plants due to their different photosynthetic physiology. This may be observed in C3 plants such as wheat, rice, beans, most fruits and vegetables which exhibit a higher δ¹³C value than C4 plants such as corn, sugar cane and sorghum (“Stable Isotope Ratios as biomarkers of diet for health research” by D. M. O'Brien, Annual Reviews (www.annualreviews.org), 2015). In one embodiment, the UDCA shows a δ¹³C value that is different from the δ¹³C value of UDCA obtained from animal sources. In a further embodiment, the UDCA shows a δ¹³C value that is different from the δ¹³C value of UDCA obtained from animal sources.

The UDCA carbons preferably are derived predominantly from plant sources, with only a minor amount (if any) of carbons derived from non-plant sources. Thus, in various preferred embodiments the carbons in the UDCA comprise greater than 80% or 90% plant derived carbons, with the remainder derived from non-plant sources. More particularly, the carbons in the steroidal rings are preferably 100% derived from plant sources, while any appended moieties may be derived from non-plant sources.

### Ketoreductase Enzymes

Preferred ketoreductases have the sequences described in the examples hereto. The invention further contemplates ketoreductases having substantial identity with the sequences described in the examples, with “substantial identity” as defined herein. Thus, the invention further contemplates ketoreductases having greater than 85% identity, 90% identity, 95% identity, or 98%, to a reference sequence over a comparison window spanning 50 amino acids, 100 amino acids, 150 amino acids, 200 amino acids, 250 amino acids, or the entire amino acid sequence.

Ketoreductase enzymes having improved properties can be obtained by mutating the genetic material encoding the ketoreductase enzyme and identifying polynucleotides that express engineered enzymes with a desired property. These non-naturally occurring ketoreductases can be generated by various well-known techniques, such as in vitro mutagenesis or directed evolution. In some embodiments, directed evolution is an attractive method for generating engineered enzymes because of the relative ease of generating mutations throughout the whole of the gene coding for the polypeptide, as well as providing the ability to take previously mutated polynucleotides and subjecting them to additional cycles of mutagenesis and/or recombination to obtain further improvements in a selected enzyme property. Subjecting the whole gene to mutagenesis can reduce the bias that may result from restricting the changes to a limited region of the gene. It can also enhance generation of enzymes affected in different enzyme properties since distantly spaced parts of the enzyme may play a role in various aspects of enzyme function.

In mutagenesis and directed evolution, the parent or reference polynucleotide encoding the naturally occurring or wild type ketoreductase is subjected to mutagenic processes, for example random mutagenesis and recombination, to introduce mutations into the polynucleotide. The mutated polynucleotide is expressed and translated, thereby generating engineered ketoreductase enzymes with modifications to the polypeptide. As used herein, “modifications” include amino acid substitutions, deletions, and insertions. Any one or a combination of modifications can be introduced into the naturally occurring enzymatically active polypeptide to generate engineered enzymes, which are then screened by various methods to identify polypeptides, and corresponding polynucleotides, having a desired improvement in a specific enzyme property.

In one embodiment, the ketoreductase is not from Clostridium absonum.

### Ketoreductase Environment

The ketoreductase enzymes may be present within a cell, in the cellular medium, on an immobilized substrate, or in other forms, such as lysates and extracts of cells recombinantly designed to express the enzyme, or isolated preparations. The term “isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis).

In some embodiments, the isolated ketoreductase polypeptide is a substantially pure polypeptide composition. The term “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure ketoreductase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

### Encoding Polynucleotide

An isolated polynucleotide encoding a ketoreductase polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3^{rd} Ed., Cold Spring Harbor Laboratory Press; and Current Protocols in Molecular Biology, Ausubel. F. ed., Greene Pub. Associates, 1998, updates to 2006.

Thus, in another aspect, the present disclosure is also directed to a recombinant expression vector comprising a polynucleotide encoding a ketoreductase polypeptide or a variant thereof, and one or more expression regulating regions such as a promoter and a terminator, a replication origin, etc., depending on the type of hosts into which they are to be introduced. The various nucleic acid and control sequences may be joined together to produce a recombinant expression vector which may include one or more convenient restriction sites to allow for insertion or substitution of the nucleic acid sequence encoding the polypeptide at such sites. Alternatively, the nucleic acid sequence of the present disclosure may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell may be used.

The term “control sequence” is defined herein to include all components, which are necessary or advantageous for the expression of a polypeptide of the present disclosure. Each control sequence may be native or foreign to the nucleic acid sequence encoding the polypeptide. Such control sequences include, but are not limited to, a leader, polyadenylation sequence, pro-peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

The term “operably linked” is defined herein is a configuration in which a control sequence is appropriately placed at a position relative to the coding sequence of the DNA sequence such that the control sequence directs the expression of a polynucleotide and/or polypeptide. The control sequence may be an appropriate promoter sequence. The “promoter sequence” is a nucleic acid sequence that is recognized by a host cell for expression of the coding region. The promoter sequence contains transcriptional control sequences, which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

### Host Cells for Expression of Ketoreductase Polypeptides

In another aspect, the present disclosure provides a host cell comprising a polynucleotide encoding a ketoreductase polypeptide of the present disclosure, the polynucleotide being operatively linked to one or more control sequences for expression of the ketoreductase enzyme in the host cell. Host cells for use in expressing the KRED polypeptides encoded by the expression vectors of the present invention are well known in the art and include but are not limited to, bacterial cells, such as E. coli cells; fungal cells, such as yeast cells (e.g., Saccharomyces cerevisiae or Pichia pastoris). In one particular embodiment, the process of the current invention is carried out with whole cells that express the 3-ketoreductase, or an extract or lysate of such cells, wherein the whole cells or extract or lysate of such whole cells are selected from Escherichia coli, Pichia pastoris or Saccharomyces cerevisiae. Appropriate culture mediums and growth conditions for the above-described host cells are well known in the art.

Polynucleotides for expression of the ketoreductase may be introduced into cells by various methods known in the art. For the bacterial systems and yeasts described herein, the typical process is by transformation (e.g. electroporation or calcium chloride mediated) or conjugation, or sometimes protoplast fusion. Various methods for introducing polynucleotides into cells will be apparent to the skilled artisan.

### Cofactors

As is known by those of skill in the art, ketoreductase-catalyzed reduction reactions typically require a cofactor. As used herein, the term “cofactor” refers to a non-protein compound that operates in combination with a ketoreductase enzyme. Cofactors suitable for use with the ketoreductase enzymes described herein include, but are not limited to, NADP⁺ (nicotinamide adenine dinucleotide phosphate), NADPH (the reduced form of NADP⁺), NAD⁺ (nicotinamide adenine dinucleotide) and NADH (the reduced form of NAD⁺). The weight ratio of the cofactor to the 3-ketoreductase is commonly from about 10:1 to 100:1.

The following equation illustrates an embodiment of a ketoreductase catalyzed reduction reaction utilizing NADH or NADPH as a cofactor, which are represented as alternatives by the designation NAD(P)H:

3-keto-sterol+NAD(P)H+H⁺+KRED [symbol] 3-beta-hydroxy-sterol+NAD(P)⁺

The reduced NAD(P)H form can be optionally regenerated from the oxidized NAD(P) form using a cofactor regeneration system. The term “cofactor regeneration system” refers to a set of reactants that participate in a reaction that reduces the oxidized form of the cofactor (e.g., NADP⁺ to NADPH). Cofactors oxidized by the ketoreductase-catalyzed reduction of the 3-keto-sterol are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst, that catalyzes the reduction of the oxidized form of the cofactor by the reductant.

Exemplary cofactor regeneration systems that may be employed include, but are not limited to, glucose and glucose dehydrogenase, formate and formate dehydrogenase, glucose-6-phosphate and glucose-6-phosphate dehydrogenase, a secondary (e.g., isopropanol) alcohol and secondary alcohol dehydrogenase, phosphite and phosphite dehydrogenase, molecular hydrogen and dehydrogenase, and the like. These systems may be used in combination with either NADP⁺/NADPH or NAD⁺/NADH as the cofactor.

In some embodiments, when the process is carried out using whole cells of the host organism, the whole cell may natively provide the cofactor. Alternatively or in combination, the cell may natively or recombinantly provide the cofactor.

### Reaction Conditions

In carrying out the stereoselective reductions described herein, the ketoreductase enzyme, and any enzymes comprising the optional cofactor regeneration system, may be added to the reaction mixture in the form of the purified enzymes (including immobilized variants), whole cells transformed with gene(s) encoding the enzymes, and/or cell extracts and/or lysates of such cells. The gene(s) encoding the engineered ketoreductase enzyme and the optional cofactor regeneration enzymes can be transformed into host cells separately or together into the same host cell.

For example, in some embodiments one set of host cells can be transformed with gene(s) encoding the ketoreductase enzyme and another set can be transformed with gene(s) encoding the cofactor regeneration enzymes. Both sets of transformed cells can be utilized together in the reaction mixture in the form of whole cells, or in the form of lysates or extracts derived therefrom. In other embodiments, a host cell can be transformed with gene(s) encoding both the engineered ketoreductase enzyme and the cofactor regeneration enzymes.

Whole cells transformed with gene(s) encoding the ketoreductase enzyme and/or the optional cofactor regeneration enzymes, or cell extracts and/or lysates thereof, may be employed in a variety of different forms, including solid (e.g., lyophilized, spray-dried, immobilized, and the like) or semisolid (e.g., a crude paste). The cell extracts or cell lysates may be partially purified by precipitation (ammonium sulfate, polyethyleneimine, heat treatment or the like), followed by a desalting procedure prior to lyophilization (e.g., ultrafiltration, dialysis, and the like).

The quantities of reactants used in the reduction reaction will generally vary depending on the quantities of ketoreductase substrate employed. The following guidelines can be used to determine the amounts of ketoreductase, cofactor, and optional cofactor regeneration system to use. Generally, 3-keto-sterol substrates are employed at a concentration of about 20 to 300 grams/liter using from about 50 mg/liter to about 5 g/liter of ketoreductase and about 10 mg/liter to about 150 mg/liter of cofactor. The weight ratio of Compound 1 or Compound 2 to the 3-ketoreductase in the reaction mixture is commonly from about 10:1 to 200:1. Those having ordinary skill in the art will readily understand how to vary these quantities to tailor them to the desired level of productivity and scale of production.

Appropriate quantities of optional cofactor regeneration system may be readily determined by routine experimentation based on the amount of cofactor and/or ketoreductase utilized. In general, the reductant (e.g., glucose, formate, isopropanol) is utilized at levels above the equimolar level of ketoreductase substrate to achieve essentially complete or near complete conversion of the ketoreductase substrate.

The order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor regeneration system, cofactor, ketoreductase, and ketoreductase substrate may be added first to the solvent. Preferably, however, the enzyme preparation is added last.

Suitable conditions for carrying out the ketoreductase-catalyzed reduction reactions described herein include a wide variety of conditions including contacting the ketoreductase enzyme and substrate at an experimental pH and temperature and detecting product, for example, using the methods described in the Examples provided herein.

The ketoreductase-catalyzed reduction reactions described herein are generally carried out in a solvent. Suitable solvents include water, organic solvents (e.g., ethyl acetate, butyl acetate, 1-octanol, heptane, octane, methyl t-butyl ether (MTBE), toluene, and the like), ionic liquids (e.g., 1-ethyl 4-methylimidazolium tetrafluoroborate, 1-butyl-3-methylimidazolium tetrafluoroborate, 1-butyl-3-methylimidazolium hexafluorophosphate, and the like). In some embodiments, aqueous solvents, including water and aqueous co-solvent systems, are used. The solvent system is preferably greater than 50%, 75%, 90%, 95%, or 98% water, and in one embodiment is 100% water.

During the course of the reduction reactions, the pH of the reaction mixture may change. The pH of the reaction mixture may be maintained at a desired pH or within a desired pH range by the addition of an acid or a base during the course of the reaction. Alternatively, the pH may be controlled by using a solvent that comprises a buffer. Suitable buffers to maintain desired pH ranges are known in the art and include, for example, phosphate buffer, triethanolamine buffer, and the like. Combinations of buffering and acid or base addition may also be used.

The ketoreductase catalyzed reduction is typically carried out at a temperature in the range of from about 15° C. to about 75° C. For some embodiments, the reaction is carried out at a temperature in the range of from about 20° C. to about 55° C. In still other embodiments, it is carried out at a temperature in the range of from about 20° C. to about 45° C. The reaction may also be carried out under ambient conditions.

The reduction reaction is generally allowed to proceed until essentially complete, or near complete, reduction of substrate is obtained. Reduction of substrate to product can be monitored using known methods by detecting substrate and/or product. Suitable methods include gas chromatography, HPLC, TLC, and the like. Conversion yields of the sterol reduction product generated in the reaction mixture are generally greater than about 50%, may also be greater than about 60%, may also be greater than about 70%, may also be greater than about 80%, may also be greater than 90%, and can even be greater than about 97% or 99%.

The keto-reduction product can be recovered from the reaction mixture and optionally further purified using methods that are known to those of skill in the art. Chromatographic techniques for isolation of the ketoreduction products include both reverse-phase and normal-phase chromatography. A preferred method for product purification involves extraction into an organic solvent and subsequent crystallization.

### Dosage Forms Routes of Administration

Pharmaceutical compositions (which by definition includes dietary supplements and other manufactured dosage forms) for preventing and/or treating a subject are further provided comprising a therapeutically effective amount of UDCA, or a pharmaceutically acceptable salt thereof, and one or more pharmaceutically acceptable excipients. A “pharmaceutically acceptable excipient” is one that is not biologically or otherwise undesirable, i.e., the material can be administered to a subject without causing any undesirable biological effects or interacting in a deleterious manner with any of the other components of the pharmaceutical composition in which it is contained. The carrier can be selected to minimize any degradation of the active ingredient and to minimize any adverse side effects in the subject, as would be well known to one of skill in the art. The carrier can be a solid, a liquid, or both.

The disclosed compounds can be administered by any suitable route, preferably in the form of a pharmaceutical composition adapted to such a route, and in a dose effective for the treatment or prevention intended. In a preferred embodiment, the active compounds and compositions, are administered orally. Suitable carriers and their formulations are described in Remington: The Science and Practice of Pharmacy (19th ed.) ed. A. R. Gennaro, Mack Publishing Company, Easton, Pa., 1995. Oral administration of a solid dose form can be, for example, presented in discrete units, such as hard or soft capsules, pills, sachets, lozenges, or tablets, each containing a predetermined amount of at least one of the disclosed compound or compositions. In some forms, the oral administration can be in a powder or granule form. In the case of capsules, tablets, and pills, the dosage forms also can comprise buffering agents or can be prepared with enteric coatings.

Preferred embodiments 1-46 are described below:

[Embodiment 1]A compound selected from ursodeoxycholic acid of formula I:

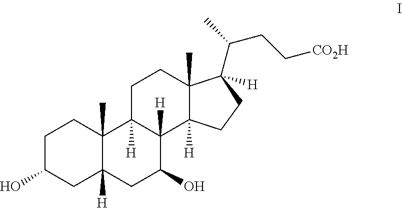

and its pharmaceutically acceptable salts comprising a δ¹³C value corresponding to a plant derived molecule, preferably comprising less than −15‰, −17.5‰, −20‰, −22.5‰, or −25% ‰ δ13C relative to VPDB, and an impurity profile characterized by: (a) less than 5%, 3%, 1%, 0.5%, 0.1%, 0.05%, 0.03%, or 0.01% of any 3β-hydroxysteroids; (b) less than 5%, 3%, 1%, 0.5%, 0.1%, 0.05%, 0.03%, or 0.01% of any 5α-steroids; or (c) less than 5%, 3%, 1%, 0.5%, 0.1%, 0.05%, 0.03%, or 0.01% of any 7α-hydroxysteroids.

[Embodiment 2] The compound of Embodiment 1 comprising an impurity profile characterized by less than 0.5%, 0.1%, 0.05%, 0.03%, or 0.01% of any 3β-hydroxysteroids and less than 0.5%, 0.1%, 0.05%, 0.03%, or 0.01% of any 7α-hydroxysteroids, made by a process that goes through a DKCA intermediate, comprising: (a) contacting the DKCA with a 3α-hydroxysteroid dehydrogenase to stereo-selectively reduce the DKCA to a 3α hydroxy intermediate, and contacting the 3α-hydroxy intermediate with a 7β-hydroxysteroid dehydrogenase to stereo-selectively reduce the 3α hydroxy intermediate to UDCA; (b) contacting the DKCA with a 7β-hydroxysteroid dehydrogenase to stereo-selectively reduce the DKCA to a 7β hydroxy intermediate, and contacting the 7β hydroxy intermediate with a 3α-hydroxysteroid dehydrogenase to stereo-selectively reduce the 7β hydroxy intermediate to UDCA; or (c) simultaneously contacting the DKCA with a 3α-hydroxysteroid dehydrogenase and a 7β-hydroxysteroid dehydrogenase to stereo-selectively reduce the DKCA to UDCA.

[Embodiment 3] The compound of Embodiment 1 comprising an impurity profile characterized by less than 1.0%, 0.5%, 0.1%, 0.05%, 0.03%, or 0.01% of any 5α-steroids, made by a process that goes through a 4,5 unsaturated 3,7-diketo DKCA precursor, comprising contacting the precursor with a Pd catalyst in the presence of pyridine or a pyridine derivative, thereby hydrogenating the 4,5 double bond to produce DKCA.

[Embodiment 4] The compound of Embodiment 1 further comprising: (a) less than 5%, 3%, 1%, 0.5%, 0.1%, 0.05%, 0.03%, or 0.01% of LCA; (b) less than 5%, 3%, 1%, 0.5%, 0.1%, 0.05%, 0.03%, or 0.01% of any 3-keto, 7-hydroxysteroids; (c) less than 5%, 3%, 1%, 0.5%, 0.1%, 0.05%, 0.03%, or 0.01% of any 3-hydroxy, 7-ketosteroids; and/or (d) less than 5%, 3%, 1%, 0.5%, 0.1%, 0.05%, 0.03%, or 0.01% of DKCA.

[Embodiment 5] The compound of Embodiment 1 comprising an impurity profile characterized by: (a) less than 1% of any 3β-hydroxysteroids; (b) less than 1% of any 5α-steroids; or (c) less than 1% of any 7α-hydroxysteroids.

[Embodiment 6] The compound of Embodiment 1 comprising an impurity profile characterized by: (a) less than 0.1% of any 3β-hydroxysteroids; (b) less than 0.5% of any 5α-steroids; or (c) less than 0.1% of any 7α-hydroxysteroids.

[Embodiment 7] The compound of Embodiment 1 comprising an impurity profile characterized by: (a) less than 5%, 3%, 1%, 0.5%, 0.1%, 0.05%, 0.03%, or 0.01% of any 3β-hydroxysteroids; (b) less than 5%, 3%, 1%, 0.5%, 0.1%, 0.05%, 0.03%, or 0.01% of any 5α-steroids; and (c) less than 5%, 3%, 1%, 0.5%, 0.1%, 0.05%, 0.03%, or 0.01% of any 7α-hydroxysteroids.

[Embodiment 8] The compound of Embodiment 1 comprising an impurity profile characterized by: (a) less than 1% of any 3β-hydroxysteroids; (b) less than 1% of any 5α-steroids; and (c) less than 1% of any 7α-hydroxysteroids.

[Embodiment 9] The compound of Embodiment 1 comprising an impurity profile characterized by: (a) less than 0.1% of any 3β-hydroxysteroids; (b) less than 0.5% of any 5α-steroids; and (c) less than 0.1% of any 7α-hydroxysteroids.

[Embodiment 10] The compound of any of Embodiments 1-9 further comprising less than 3%, 2%, or 1% of impurities selected from starting materials, by-products, intermediates, and degradation products.

[Embodiment 11]A compound selected from ursodeoxycholic acid of formula I:

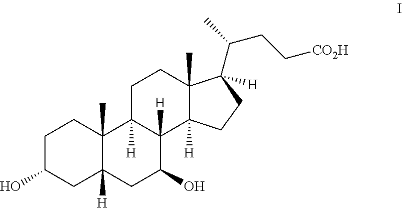

and its pharmaceutically acceptable salts comprising an impurity profile characterized by: (a) less than 0.05%, 0.03%, or 0.01% of any 3β-hydroxysteroids; or (b) less than 0.05%, 0.03%, or 0.01% of any 5α-steroids; or (c) less than 0.05%, 0.03%, or 0.01% of any 7α-steroids.

[Embodiment 12] The compound of Embodiment 11 comprising an impurity profile characterized by: (a) less than 0.01% of any 3β-hydroxysteroids; or (b) less than 0.01% of any 5α-steroids; or (c) less than 0.01% of any 7α-hydroxysteroids.

[Embodiment 13] The compound of Embodiment 11 comprising an impurity profile characterized by: (a) less than 0.05%, 0.03%, or 0.01% of any 3β-hydroxysteroids; (b) less than 0.05%, 0.03%, or 0.01% of any 5α-steroids; and (c) less than 0.05%, 0.03%, or 0.01% of any 7α-hydroxysteroids.

[Embodiment 14] The compound of Embodiment 11 comprising an impurity profile characterized by: (a) less than 0.01% of any 3β-hydroxysteroids; (b) less than 0.01% of any 5α-steroids; and (c) less than 0.01% of any 7α-hydroxysteroids.

[Embodiment 15] The compound of Embodiment 11 comprising an impurity profile characterized by: (a) less than 5%, 3%, 1%, 0.5%, 0.1%, 0.05%, 0.03%, or 0.01% of LCA; (b) less than 5%, 3%, 1%, 0.5%, 0.1%, 0.05%, 0.03%, or 0.01% of any 3-keto, 7-hydroxysteroids; (c) less than 5%, 3%, 1%, 0.5%, 0.1%, 0.05%, 0.03%, or 0.01% of any 3-hydroxy, 7-ketosteroids; (d) less than 5%, 3%, 1%, 0.5%, 0.1%, 0.05%, 0.03%, or 0.01% of DKCA; (e) or a combination thereof.

[Embodiment 16] The compound of any of Embodiments 11-15 comprising less than 1% or 0.5% of impurities selected from starting materials, by-products, intermediates, and degradation products.

[Embodiment 17] The compound of any of Embodiments 11-15 comprising a δ¹³C value corresponding to a plant derived molecule, preferably comprising less than −20%, −22.5%, or −25% ‰ δ13C relative to VPDB.

[Embodiment 18] The compound of any of Embodiments 1-17 in an isolated state.

[Embodiment 19]A pharmaceutical composition comprising the compound of any of Embodiments 1-18 and one or more pharmaceutically acceptable excipients.

[Embodiment 20]A method of making a UDCA pharmaceutical dosage form comprising admixing the compound of any of Embodiments 1-18 with one or more pharmaceutically acceptable excipients to form an admixture and processing the admixture into a finished dosage form, preferably by compressing the admixture into a tablet or filling the admixture into a capsule or sachet.

[Embodiment 21]A method of producing the compound of any of Embodiments 1-18 that goes through a DKCA intermediate, comprising: (a) contacting the DKCA with a 3α-hydroxysteroid dehydrogenase to stereo-selectively reduce the DKCA to a 3α hydroxy intermediate, and contacting the 3α hydroxy intermediate with a 7β-hydroxysteroid dehydrogenase to stereo-selectively reduce the 3α hydroxy intermediate to UDCA; or (b) contacting the DKCA with a 7β-hydroxysteroid dehydrogenase to stereo-selectively reduce the DKCA to a 7β hydroxy intermediate, and contacting the 7β hydroxy intermediate with a 3α-hydroxysteroid dehydrogenase to stereo-selectively reduce the 7β hydroxy intermediate to UDCA; or (c) simultaneously contacting the DKCA with a 3α-hydroxysteroid dehydrogenase and a 7β-hydroxysteroid dehydrogenase to stereo-selectively reduce the DKCA to UDCA.

[Embodiment 22] The method of Embodiment 21, carried out with whole cells that express the hydroxysteroid dehydrogenase, the 7β-hydroxysteroid dehydrogenase, or both, or an extract or lysate of such cells, wherein the whole cells or extract or lysate of such whole cells are selected from native or recombinant bacteria or yeast, preferably Escherichia coli, Pichia pastoris or Saccharomyces cerevisiae.

[Embodiment 23] The method of Embodiment 21 or 22, wherein the DKCA is: (a) provided as or derived from an ethylenediamine salt of 3,7-DKCA, optionally a crystalline form defined by Pattern 6-D, (b) provided as or derived from a tert-butylamine salt of 3,7-DKCA, optionally a crystalline form defined by Pattern 9-A, or (c) provided as or derived from a diisopropylamine salt of 3,7-DKCA, optionally a crystalline form defined by Pattern 10-A, further optionally comprising converting the salt to a free acid.

[Embodiment 24]A method of producing the compound of any of Embodiments 1-18, made by a process that goes through a 4,5 unsaturated 3,7-diketo DKCA precursor, comprising contacting a 4,5 unsaturated 3,7-diketo DKCA precursor with a Pd catalyst in the presence of pyridine or a pyridine derivative, thereby hydrogenating the 4,5 double bond to produce DKCA.

[Embodiment 25] The method of any of Embodiments 21-24, further comprising isolating the UDCA.

[Embodiment 26] The method of any of Embodiments 21-25, further comprising admixing the UDCA with one or more pharmaceutically acceptable excipients to form an admixture and processing the admixture into a finished dosage form, optionally by compressing the admixture into a tablet or filling the admixture into a capsule or sachet.

[Embodiment 27] An ethylenediamine salt of 3,7-DKCA.

[Embodiment 28] The ethylenediamine salt of 3,7-DKCA of Embodiment 27 having crystalline form Pattern 6-D defined by: (a) an XRPD pattern comprising at least one, two, or three peaks in terms of 2θ, selected from the group consisting of 5.81, 8.69, 9.95, 10.92, 11.60, 13.08, 13.78, 14.59, 16.03, 16.51, 25.11, 27.42, 28.82, 30.24, 33.35, and 38.22°±0.2°, or (b) an XRPD pattern substantially as depicted in FIG. 4.

[Embodiment 29]A tert-butylamine salt of 3,7-DKCA.

[Embodiment 30] The tert-butylamine salt of 3,7-DKCA of Embodiment 29 having crystalline form Pattern 9-A defined by:
(a) an XRPD pattern comprising at least one, two, or three peaks in terms of 20, selected from the group consisting of 4.83, 8.77, 13.35 15.56, 16.03, 20.54, 22.05, 23.53, 24.75, 29.93, 30.40, and 31.970±0.2°, or (b) an XRPD pattern substantially as depicted in FIG. 3.

[Embodiment 31]A diisopropylamine salt of 3,7-DKCA.

[Embodiment 32] The diisopropylamine salt of 3,7-DKCA of Embodiment 31 having crystalline form Pattern 10-A defined by (a) an XRPD pattern comprising at least one, two, or three peaks in terms of 20, selected from the group consisting of 5.85, 6.29, 9.05, 12.58, 14.17, 16.09, 18.13, 18.47, 18.89, 20.49, 21.48, 24.75, 25.27, 28.65, 30.21, 31.82, 34.78, and 37.44°±0.2°, or (b) has an XRPD pattern substantially as depicted in FIG. 5.

[Embodiment 33] The compound of any of Embodiments 27-32 in an isolated state.

[Embodiment 34] Ursodeoxycholic acid or a pharmaceutically acceptable salt thereof comprising a δ¹³C value corresponding to a plant derived molecule of less than −15% ‰ relative to VPDB, and an impurity profile characterized by less than 3% of any 5α-steroids.

[Embodiment 35] Ursodeoxycholic acid or a pharmaceutically acceptable salt thereof comprising a δ¹³C value corresponding to a plant derived molecule of less than −20% ‰ relative to VPDB, and an impurity profile characterized by less than 1% of any 5α-steroids.

[Embodiment 36] Ursodeoxycholic acid or a pharmaceutically acceptable salt thereof comprising a δ¹³C value corresponding to a plant derived molecule of less than −20% ‰ relative to VPDB, and an impurity profile characterized by less than 0.5% of any 5α-steroids.

[Embodiment 37] Ursodeoxycholic acid or a pharmaceutically acceptable salt thereof comprising a δ¹³C value corresponding to a plant derived molecule of less than −20% ‰ relative to VPDB, and an impurity profile characterized by less than 0.1% of any 5α-steroids.

[Embodiment 38] Ursodeoxycholic acid or a pharmaceutically acceptable salt thereof comprising a δ¹³C value corresponding to a plant derived molecule of less than −20% ‰ relative to VPDB, and an impurity profile characterized by less than 0.05% of any 5α-steroids.

[Embodiment 39] The compound of any of Embodiments 34-38 comprising less than 1% of any 3β-hydroxysteroids and less than 1% of any 7α-hydroxysteroids.

[Embodiment 40] The compound of any of Embodiments comprising less than 0.5% of any 3β-hydroxysteroids and less than 0.5% of any 7α-hydroxysteroids.

[Embodiment 41] The compound of any of Embodiments comprising less than 0.1% of any 3β-hydroxysteroids and less than 0.1% of any 7α-hydroxysteroids.

[Embodiment 42] The compound of any of Embodiments comprising less than 0.05% of any 3β-hydroxysteroids and less than 0.05% of any 7α-hydroxysteroids.

[Embodiment 43] The compound of any of Embodiments comprising less than 0.03% of any 3β-hydroxysteroids and less than 0.03% of any 7α-hydroxysteroids.

[Embodiment 44] The compound of any of Embodiments 34-43 comprising a δ¹³C value corresponding to a plant derived molecule of less than −22.5‰, or −25‰ δ13C relative to VPDB.

[Embodiment 45] The compound of any of Embodiments 34-44 in an isolated state.

[Embodiment 46]A pharmaceutical composition comprising the compound of any of Embodiments 34-44 and one or more pharmaceutically acceptable excipients.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1. Preparation of KCEA from Bisnoralcohol

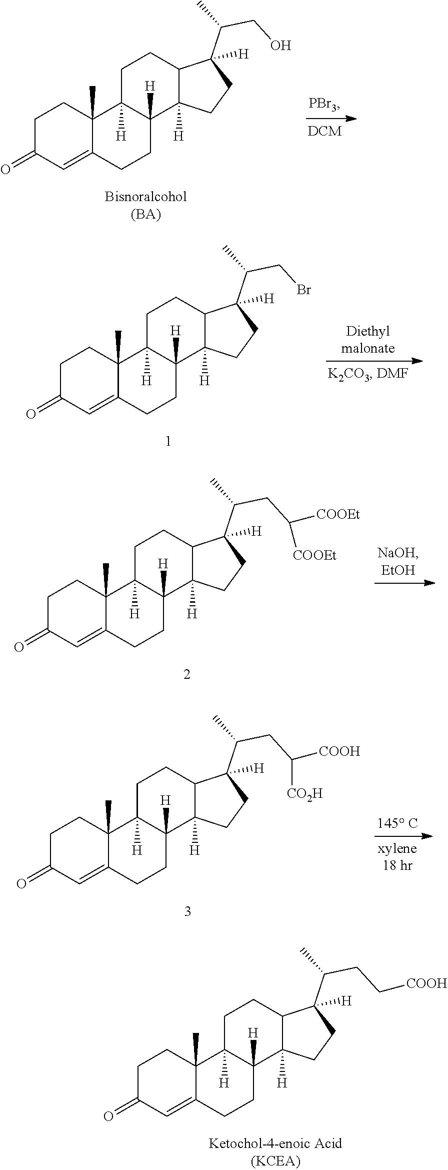

### Bromination of Bisnoralcohol:

To a stirred solution of bisnoralcohol (BA, 1 g, 3.02 mmol) in dichloromethane (DCM, 20 mL) was added PBr₃ (0.34 mL, 3.63 mmol) at 0° C. The mixture was warmed to room temperature and stirred for 3 hr, at which point TLC analysis showed complete conversion of starting material. The reaction mixture was quenched using ice water (10 mL), stirred for 15 min and the layers were separated. The aqueous layer was extracted in DCM (10 mL) and the combined organic phase was concentrated under reduced pressure to afford compound 1 as a yellow gummy oil (crude yield 1.2 g). ¹H NMR (400 MHz, DMSO-d6): δ 5.61 (s, 1H), 3.56-3.51 (m, 1H), 3.45 (dd, J=2.1 Hz and 1.1 Hz, 1H), 2.46-2.32 (m, 2H), 2.26-2.10 (m, 2H), 199-1.89 (m, 3H), 1.82-1.70 (m, 2H), 1.70-0.82 (m, 18H), 0.70 (s, 3H) ppm.

Alkylation of Diethyl Malonate with Compound 1:

To a stirred solution of compound 1 (0.5 g, 1.27 mmol) in DMF (10 mL) was added diethyl malonate (0.58 mL, 3.812 mmol) at room temperature under N₂ atmosphere. To this solution was added K₂CO₃ (526 mg, 3.812 mmol) followed by catalytic amounts of tetrabutylammonium hydrogen sulfate (TBAHS, 43 mg, 0.127 mmol). The reaction mixture was stirred at 75-80° C. for 48 hr and TLC analysis suggested complete conversion of starting material. After completion, the reaction mixture was quenched with ice water (10 mL) and the product was extracted using ethyl acetate (2×25 mL). The combined organic layer was washed with water (20 mL) and the organic phase was concentrated under reduced pressure to obtain compound 2 as gummy oil (crude yield 700 mg). ¹H NMR (400 MHz, DMSO-d6): δ 5.61 (s, 1H), 4.0-4.20 (m, 4H), 3.50-3.42 (m, 1H), 2.43-2.30 (m, 2H), 2.27-2.10 (m, 2H), 2.11-1.90 (m, 3H), 1.89-1.70 (m, 2H), 1.62-0.80 (m, 27H), 0.63 (s, 3H) ppm. Mass analysis: m/z 473.40 [M+H]⁺ was observed.

### Hydrolysis of Compound 2:

To a stirred solution of compound 2 (12 g, 25.38 mmol) in ethanol (120 mL) was added aq. potassium hydroxide solution (7.06 g in 120 mL water, 0.127 mol) at room temperature. The reaction mixture was heated to reflux for 2 hr and TLC analysis showed complete conversion of starting material. The ethanol was evaporated under reduced pressure and the solution was diluted with water (60 mL). The mixture was washed with DCM (60 mL, to remove impurities) and the pH of the aq. layer was adjusted to ˜2 by using 6N HCl. The product was extracted using EtOAc (2×50 mL) and concentrated to dryness to afford compound 3 as a yellow solid (9.5 g).

### Decarboxylation of Compound 3:

To a 50 mL single neck round bottom flask was added compound 3 (1 g, 2.4 mmol.) in o-xylene (5 mL). The mixture was heated to reflux for 18 h and TLC analysis showed complete conversion of starting material. o-Xylene was removed under vacuum and the residue was treated with petroleum ether and the solid was filtered. The wet cake was washed with petroleum ether and dried under vacuum to afford KCEA as an off-white solid (0.5 g). ¹H NMR (400 MHz, DMSO-D6): δ 11.95 (bs, 1H), 5.62 (s, 1H), 2.44-2.34 (m, 2H), 2.28-2.06 (m, 5H), 2.0-1.91 (m, 2H), 1.87-1.74 (m, 2H), 1.72-0.81 (m, 20H), 0.69 (s, 3H) ppm.

### Example 2. Preparation of 3,7-DKCA from KCEA

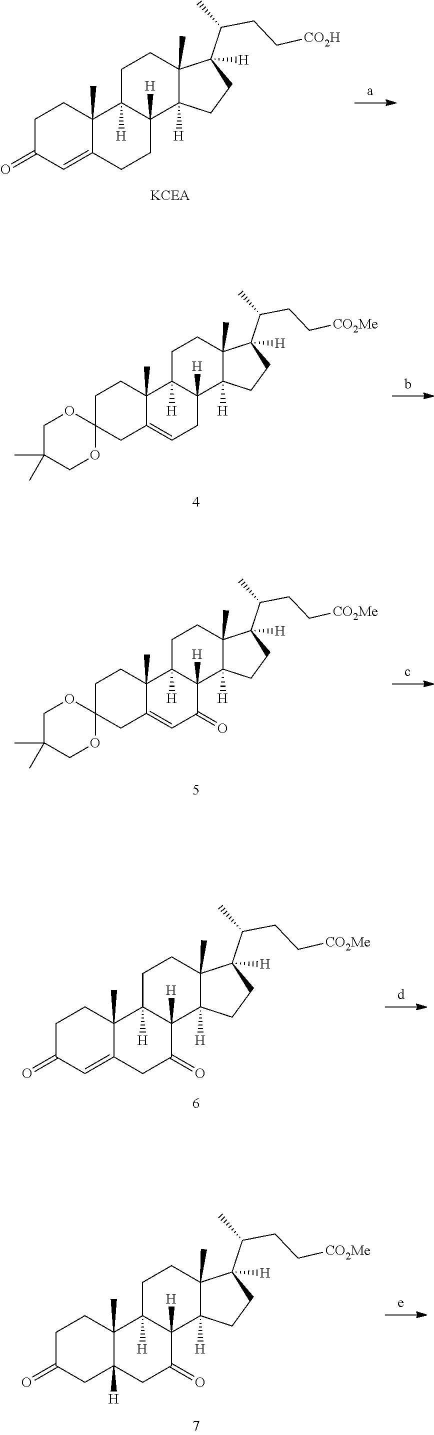

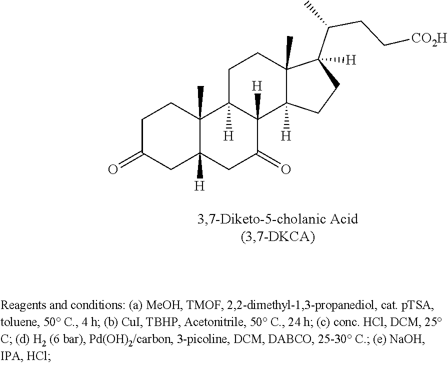

Preparation of Compound 4 from KCEA:

A 250 mL round bottom flask equipped with a stirring bar and reflux condenser was charged with toluene (90 mL), methanol (10 mL) and KCEA (10 g, 26.842 mmol). The resulting solution was inerted with nitrogen and then trimethyl orthoformate (8.8 mL, 3 equiv.) and p-toluenesulfonic acid (0.5 g, 0.1 equiv.) were added sequentially. The resulting mixture was stirred at 50-55° C. for 1 hr. The pressure was then reduced and ˜20 mL of solvent was removed via distillation. 2,2-Dimethylpropane-1,3-diol (22.3 g, 8 equiv.) and p-toluenesulfonic acid (0.5 g, 0.1 equiv.) were added and the reaction was continued for another 3 hr. At this point the mixture was cooled to 5° C. in an ice bath and treated with aqueous sodium acetate solution (30 g in 150 mL water). The mixture was stirred for 1 h at 5° C. and the resulting suspension was filtered to obtain crude product. This was purified further by silica gel chromatography to obtain Compound 4 as a white solid. (7.4 g). ¹H NMR (400 MHz, CDCl3) δ 5.38-5.33 (m, 1H), 3.68 (s, 3H), 3.60, 3.50 (ABq, 2H, J_{AB}=11.2 Hz), 3.49-3.43 (m, 2 h), 2.61-0.91 (m, 37H), 0.69 (s, 3H); ESIMS for C₃₀H₄₈O₄ m/z 473.6 [M+H]⁺.

### Oxidation of Compound 4 to Compound 5:

To a solution of compound 4 (10 g) in 4:1 acetone/DCM (200 mL) at 25-35° C. was added N-hydroxyphthalimide (NHPI, 1.73 g), benzoyl peroxide (0.05 g), copper iodide (CuI, 0.04 g) and water (0.4 mL). The mixture was heated to 40-45° C. and air was bubbled through the mixture for 7 hr. The mixture was then cooled to 25-30° C. and the air bubbling was replaced with 98% oxygen bubbling. GC analysis after 36 hr total time indicated only 1.5% of compound 4 remains.

The reaction mixture was concentrated to a residue under vacuum and diluted with DCM (20 mL). The resulting slurry was filtered to remove NHPI. The filtrate was concentrated to −15 mL and solvent was swapped with MeOH using vacuum distillation. The mixture was diluted with MeOH (25 mL), cooled to 5-10° C. and filtered. The filter cake was washed with cold MeOH (5 mL) and dried under vacuum at 40-45° C. to afford 7.9 g of compound 5 as a light-green solid.

### Hydrolysis of Compound 5 to Compound 6:

To a solution of compound 5 (5 g) in DCM (75 mL) at 10-15° C. was added 32% conc. HCl (25 mL). The mixture was allowed to warm to 25-30° C. and held for 1.5 hr. Then the reaction mixture was diluted with water (50 mL) and the phases were separated. The aqueous layer was extracted with DCM (25 mL) and the combined DCM phases washed with water (25 mL). The DCM was treated with activated carbon (0.25 g), held for 0.25 hr and filtered over filter-aid. The filter-aid cake was washed with DCM (15 mL) and concentrated to 5-10 mL under vacuum. The residue was diluted with n-heptane (25 mL) and concentrated again to 5-10 mL. The resulting mixture was diluted with n-heptane (25 mL), cooled to 5-10° C., held for 0.5 hr and filtered. The filter cake was washed with cold n-heptane (2.5 mL) and dried under vacuum at 40-45° C. to afford 3.8 g of compound 6 as a light-orange solid.

### Hydrogenation of Compound 6 to Compound 7:

Compound 6 (180 g), dichloromethane (DCM; 45 mL) and 3-picoline (1035 mL) were combined in a 2-liter autoclave. Diazabicyclo[2.2.2]octane (DABCO; 50.4 g) and 20% Pd(OH)₂ (50% water-wet, 7.2 g) were added. The resulting mixture was stirred at 26° C. under hydrogen gas at 6 bar pressure for 22 hr. The catalyst was then removed by filtration. The solid catalyst was washed with DCM (720 mL) and the filtrate was concentrated under vacuum to remove DCM. Water (1000 mL) was added and the mixture was concentrated under vacuum at 60° C. until the total volume was ˜360 mL. Toluene (1300 mL) was added the resulting solution was washed twice with 3N HCl (2×630 mL). The aqueous washes are combined and extracted with toluene (500 mL).

The toluene fractions were combined and washed with 3N HCl (255 mL) and then distilled under vacuum to ˜360 mL. 10% Aqueous ethanol (900 mL) was added and the solution was concentrated under vacuum to ˜360 mL. Additional 10% aqueous ethanol (900 mL) was added and again the mixture was concentrated under vacuum to ˜360 mL. Additional 10% aqueous ethanol (680 mL) was added and the mixture was cooled to 0-5° C. The slurry was filtered and the cake was washed with chilled (5-10° C.) 10% aqueous ethanol (85 mL). The cake was then dried under vacuum at 40-45° C. to provide 132.4 g (73.2% yield) of compound 7 as an off-white solid.

The solid was combined with additional lots of compound 7 to give 257 g. This was dissolved in DCM (514 mL) and 10% aqueous ethanol (1030 mL) was added. The resulting mixture was distilled under vacuum to a volume of ˜500 mL, and then additional 10% aqueous ethanol (1030 mL) was added. After concentrating under vacuum again to −500 mL, additional 10% aqueous ethanol (1030 mL) was added. The mixture was cooled to 0-5° C. and filtered, and the cake was washed with chilled (5-10° C.) 10% aqueous ethanol (125 mL). The cake was then dried under vacuum at 40-45° C. to provide 238 g (92.6% recovery) of compound 7 as an off-white solid.

Melting point=167° C.; Purity by CAD HPLC (w/w %)=98.7% (5α-impurity=0.37%); ¹H-NMR (400 MHz, CDCl₃): δ 3.67 (s, 3H), 2.90 (dd, J=12.8 & 6.5 Hz, 1H), 2.49 (t, J=11.4 Hz, 1H), 0.95-2.40 (m, 27H), 0.93 (d, J=6.4 Hz, 3H), 0.69 (s, 3H).

### Hydrolysis of Compound 7 to 3,7-DKCA:

To a solution of compound 7 (6 g) in IPA (30 mL/g) was added a solution of NaOH (1.5 g, 2.5 equiv) in water (30 mL) at room temperature. The reaction was warmed to 55-60° C. until it was found to be complete by TLC analysis.

The reaction mixture was concentrated to ˜30 mL to remove residual IPA and the resulting aqueous solution washed with MTBE (2×30 mL). The aqueous phase was acidified to pH 2 using 6 M HCl, leading to the formation of a slurry. After cooling to 10-15° C., the slurry was filtered, washed with water and dried under vacuum at 45-50° C. to afford 4.2 g of 3,7-DKCA as a light-brown solid. This material can be purified as described in Example 10.

### Example 3: Preparation of UDCA from 3,7-DKCA

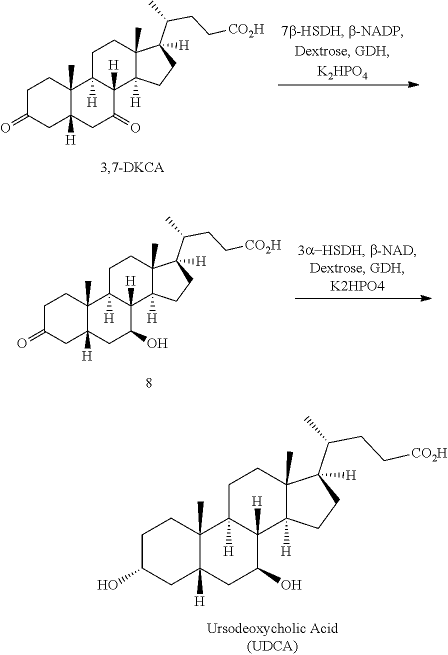

### Selective Reduction of the 7-Ketone of 3,7-DKCA to Provide Compound 8:

To a 250 mL single neck round bottom flask were added 3,7-DKCA (1 g, 2.57 mmol), dextrose (1.3 g), β-NADP (33 mg) and 250 mM K₂HPO₄ buffer (70 mL) at room temperature. The mixture was stirred for 0.5 h to get a clear solution. 7β-HSDH (66 mg) and GDH (2 mg) were added and the resulting mixture was stirred for 4 hr at room temperature. TLC analysis showed complete conversion of starting material.

The reaction mixture was quenched with 2N HCl solution until pH 3-3.5 was observed. The product was extracted in EtOAc (3×50 mL) and concentrated under reduced pressure to obtain compound 8 (1 g, as an off-white solid). ¹H NMR (400 MHz, MeOD): δ 3.49-3.58 (m, 1H), 2.70 (t, J=13.8 Hz, 1H), 2.48-2.29 (m, 3H), 2.28-2.15 (m, 2H), 2.14-2.05 (m, 5H), 1.98-1.78 (m, 7H), 1.72-0.95 (m, 15H), 0.71 (s, 3H)

### Selective Reduction of the 3-Ketone of Compound 8 to Provide UDCA:

To a 250 mL single neck round bottom flask were added compound 8 (1 g, 2.56 mmol), dextrose (1.3 g), β-NAD (33 mg) and 250 mM K₂HPO₄ buffer (70 mL) at room temperature. The mixture was stirred for 0.5 h to get a clear solution. 3α-HSDH (66 mg) and GDH (2 mg) were added and the resulting mixture was stirred for 20 hr at room temperature. TLC analysis showed complete conversion of starting material.

The reaction mixture was quenched with 2N HCl solution until the pH reached 3-3.5, and then the product was extracted in EtOAc (3×50 mL) and concentrated under reduced pressure to obtain UDCA (900 mg, as a white solid). ¹H NMR (500 MHz, MeOD): δ 3.44-3.53 (m, 2H), 2.30-2.38 (m, 1H), 2.18-2.25 (m, 1H), 2.03 (dt, J=6.5 Hz and 2.9 Hz, 1H), 1.95-1.79 (m, 5H), 1.56-0.92 (m, 26H), 0.71 (s, 3H).

While the method has been described reducing the 7-ketone on DKCA first and the 3-ketone second, the sequence of reductions using the same HSDH enzymes, reducing the 3-ketone on DKCA first, is also possible and has been carried out with similar success.

### General Methods for Examples 4-8

Isolation, handling and manipulation of DNA are carried out using standard methods (Green and Sambrook, 2012), which includes digestion with restriction enzymes, PCR, cloning techniques and transformation of bacterial cells.

Synthetic DNA is ordered from a commercial vendor, such as Eurofins, IDT, Genewiz or Twist Biosciences, as described in the examples. Genes are to be supplied in custom vectors or as linear DNA fragments, as described in the examples.

### Media

2TY medium contains 16 g/L bacto-tryptone, 10 g/L yeast extract and 5 g/L NaCl and is sterilised by autoclaving. 2TY agar additionally contains 15 g/L agar.

Low-salt LB contains 10 g/L tryptone, 5 g/L yeast extract and 5 g/L NaCl.

Seed medium contains 3 g/L yeast extract, 2.5 g/L dibasic potassium phosphate, 18 g/L vegetable peptone, 5 g/L NaCl and 10 g/L glucose.

Fermentation medium contains yeast extract 5 g/L, ammonium sulfate 1.7 g/L, dibasic potassium phosphate 7 g/L, citric acid 1 g/L, iron chloride 0.04 g/L, calcium chloride 0.03 g/L, magnesium sulfate 4.6 g/L, copper chloride 0.05 mg/L, boric acid 0.025 mg/L sodium iodide 0.5 mg/L manganese sulfate 0.5 mg/L zinc sulfate 0.1 mg/L and sodium molybdate 0.1 mg/L

Fermentation substrate feed medium contains yeast extract 5 g/L, ammonium sulfate 1.7 g/L, dibasic potassium phosphate 7 g/L, citric acid 1 g/L, iron chloride 0.04 g/L, calcium chloride 0.03 g/L, magnesium sulfate 4.6 g/L, copper chloride 0.05 mg/L, boric acid 0.025 mg/L sodium iodide 0.5 mg/L manganese sulfate 0.5 mg/L zinc sulfate 0.1 mg/L sodium molybdate 0.1 mg/L and 350 g/L glucose

### Materials

Restriction enzymes are purchased from New England Biolabs (NEB) or Promega. Media components, chemicals and PCR primers are obtained from Sigma-Aldrich (Merck).

### Example 4. Construction of an Escherichia coli Strain Capable of Expressing a Gene Encoding a 3α-Hydroxy-Steroid Dehydrogenase Enzyme from Comamonas testosterone

Plasmid pSAND150 was constructed as follows. SEQ ID NO. 1 was ordered as synthetic DNA (Integrated DNA Technologies) and amplified by PCR using primers SEQ ID NO. 2 and SEQ ID NO. 3, resulting in a 2541 bp fragment, to be used as fragment A. SEQ ID NO. 4 was ordered as synthetic DNA (Integrated DNA technologies) and amplified by PCR using primers SEQ ID NO. 5 and SEQ ID NO. 6, resulting in a 2927 bp fragment, to be used as fragment B. Fragment A was inserted into PCR-amplified fragment B using the SLiCE cloning method (Zhang et al., 2014), forming plasmid pSAND150. Correct assembly of the plasmid was verified by restriction digest and by sanger sequencing using primers SEQ ID NO. 7, SEQ ID NO. 8 and SEQ ID NO. 9.

Plasmid pSAND151, to express a gene encoding a 3α-hydroxy-steroid dehydrogenase from Comamonas testosteroni, was constructed as follows. Plasmid pSAND150 was amplified by PCR using primers SEQ ID NO. 10 and SEQ ID NO. 11, followed by digestion with restriction enzyme DpnI, to be used as the plasmid backbone. SEQ ID NO. 12 was ordered as synthetic DNA (Integrated DNA technologies) and amplified by PCR using primers SEQ ID NO. 13 and SEQ ID NO. 14. The resulting 874 bp fragment was inserted into PCR-amplified pSAND150 using the SLiCE cloning method (Zhang et al., 2014), forming plasmid pSAND151. Correct assembly of plasmid pSAND151 was verified by colony PCR primers SEQ ID NO. 7 and SEQ ID NO. 15 and by sanger sequencing using primers SEQ ID NO. 7, SEQ ID NO. 9 and SEQ ID NO. 15.

Plasmid pSAND151 was used to transform E. coli BL21(DE3) by electroporation using standard methods. The resulting strain was labelled Escherichia coli sp. SAND150.

### Example 5. Production of a 3α-Hydroxy-Steroid Dehydrogenase Enzyme

50 mL low-salt LB medium containing 12.5 μg/mL kanamycin in a 250-mL baffled Erlenmeyer flask was inoculated with E. coli sp. SAND150 and incubated at 37° C. with shaking at 250 RPM, 2.5 cm throw for 18 hours, to be used as the preculture. 4 mL of preculture was transferred to a 2 Litre baffled glass Erlenmeyer flask with 400 mL Seed medium containing kanamycin at 12.5 μg/mL. This culture was incubated at a 37° C. and rotated at 250 rpm for 7 hours, to be used as the seed culture. The cell density reached 7.03 OD₆₀₀.

60 mL seed culture was transferred to a 6.4 litre production stage bioreactor containing 2 litres of Fermentation media described in media section to achieve a starting biomass of 0.2 OD₆₀₀. The bioreactor was operated as a fed-batch variable volume fermentation at 31% to 78% volumetric space efficiency. The fermentation temperature was controlled to a constant 30° C. until induction with no back pressure. Dissolved oxygen was controlled at 30% with a control statement increasing stirrer incrementally from 200 to 1200 rpm increasing by 25 rpm when PO2 drops below setpoint activated at 10-minute intervals and a fixed manual airflow of 4 litres of air per minute. The agitation was achieved by two conventional 6-flat bladed disc turbines and the airflow was sparged via a submerged sparger. pH was controlled at 7.2 with the automatic addition of 28% ammonium hydroxide. Fermentation substrate feed was applied to the fermenter from the start of inoculation, where it received a linear rate of 19.2 mL/hr to 103.1 mL/hr over 24 hours.

The linear feed was continued until the optical density reached 59.8 OD₆₀₀ and the culture was induced by the addition of 0.5 mM Isopropyl ß-D-1-thiogalactopyranoside (IPTG) and reduction of temperature to 25° C. The substrate feed rate was then switched to an event-based feeding method for the remainder of the production, adding 9 mL shot of feed when the dissolved oxygen rose above 30%. The fermentation was harvested once 22.5 hours had passed since induction. Fermentation broth was centrifuged at 8000 rcf at 4° C., 45 minutes and 884 g of cell pellet was frozen at −80° C. Cells solids were then resuspended in 50 mM potassium phosphate buffer pH 8.0 to a concentration of 40% solids. The slurry was then mechanically lysed using a french press cell disruptor at 1500 psi with 3 passes. Bulk lysate was diluted to 3.2 litres before polyethyleneimine was added to a final concentration of 0.4%. The mixture was agitated for 10 minutes before centrifuged again at 8000 ×g for 15 minutes. The supernatant was retained, and the volume was concentrated by 37% using a 5 kDa MWCO PES filtration membrane. Retentate liquid was then dried under vacuum to create a lyophilised powder.

### Example 6. Construction of an Escherichia coli Strain Lacking Native 7α-Hydroxysteroid Dehydrogenase Activity

Plasmid pSAND152, to interrupt the hdhA gene in E. coli, was constructed as follows. SEQ ID NO. 16 was ordered as circular synthetic DNA (Twist Bioscience) and cleaved with restriction enzymes BsrGI and XbaI, to be used as the plasmid backbone. SEQ ID NO. 17 was ordered as synthetic DNA (Integrated DNA technologies) and amplified by PCR using primers SEQ ID NO. 20 and SEQ ID NO. 21. The resulting 364 bp fragment was digested with restriction enzymes BsrGI and XbaI. The digested synthetic DNA was inserted into the cleaved plasmid backbone by ligation following standard methods, forming plasmid pSAND152. Transformants were plated onto 2TY agar containing 34 μg/mL chloramphenicol. Correct assembly of plasmid pSAND152 was confirmed by sanger sequencing using primers SEQ ID NO. 18 and SEQ ID NO. 19.

Plasmid pSAND152 was used to transform E. coli BL21(DE3) by electroporation using standard methods and plated onto 2TY agar containing 50 μg/mL kanamycin and 1 mM IPTG. Agar plates were incubated at 30° C. for approximately 18 hours, followed by incubation at ambient temperature for a further 3 days. Disruption of the hdhA gene was verified by growth on 2TY agar plates containing either 50 μg/mL kanamycin or 34 μg/mL chloramphenicol, where kanamycin resistance and chloramphenicol sensitivity indicates successful disruption.

Disruption of the hdhA gene was further verified as follows. A 2829 bp DNA fragment was amplified by PCR from the genome of the transformant using primers SEQ ID NO. 22 and SEQ ID NO. 23. The amplified DNA fragment was subsequently sequenced using primers SEQ ID NO. 22 and SEQ ID NO. 23. The resulting strain was labelled Escherichia coli sp. SAND151.

### Example 7. Construction of an Escherichia coli Strain Capable of Expressing a Gene Encoding an Engineered 70-Hydroxy-Steroid Dehydrogenase Enzyme from Ruminococcus Torques

Plasmid pSAND153, to express a gene encoding a 70-hydroxy-steroid dehydrogenase, was constructed as follows. Plasmid pSAND150 was amplified by PCR using primers SEQ ID NO. 10 and SEQ ID NO. 11, followed by digestion with restriction enzyme DpnI, to be used as the plasmid backbone.

SEQ ID NO. 24 was ordered as synthetic DNA (Integrated DNA technologies) and amplified by PCR using primers SEQ ID NO. 25 and SEQ ID NO. 26. The resulting 895 bp fragment was inserted into PCR-amplified pSAND150 using the SLiCE cloning method (Zhang et al., 2014), forming plasmid pSAND153. Correct assembly of plasmid pSAND153 was verified by colony PCR and by Sanger sequencing using primers SEQ ID NO. 7, SEQ ID NO. 9 and SEQ ID NO. 15.

Plasmid pSAND154, to express a gene encoding a 70-hydroxy-steroid dehydrogenase, was constructed as follows. Plasmid pSAND153 was amplified by PCR using primers SEQ ID NO. 27 and SEQ ID NO. 28, to be used as the plasmid backbone.

SEQ ID NO. 29 was ordered as synthetic DNA (Integrated DNA Technologies) and amplified by PCR using primers SEQ ID NO. 30 and SEQ ID NO. 31. The resulting 1066 bp fragment was inserted into PCR-amplified pSAND154 using the SLiCE cloning method (Zhang et al., 2014), forming plasmid pSAND154.

Plasmid pSAND154 was used to transform E. coli sp. SAND151 by electroporation using standard methods. The resulting strain was labelled Escherichia coli sp. SAND152.

### Example 8. Production of a 7β-Hydroxy-Steroid Dehydrogenase Enzyme

50 mL low-salt LB medium containing 12.5 μg/mL kanamycin in a 250-mL baffled Erlenmeyer flask was inoculated with E. coli sp. SAND150 and incubated at 37° C. with shaking at 250 RPM, 2.5 cm throw for 18 hours, to be used as the preculture.

4 mL of preculture was transferred to a 2 Litre baffled glass Erlenmeyer flask with 400 mL Seed medium containing kanamycin at 12.5 μg/mL. This culture was incubated at a 37° C. and rotated at 250 rpm for 7 hours, to be used as the seed culture. The cell density reached 4.8 OD₆₀₀. 60 mL seed culture was transferred to a 6.4 litre production stage bioreactor containing 2 litres of Fermentation media described in media section to achieve a starting biomass of 0.14 OD₆₀₀. The bioreactor was operated as a fed-batch variable volume fermentation at 31% to 78% volumetric space efficiency. The fermentation temperature was controlled to a constant 30° C. until induction with no back pressure. Dissolved oxygen was controlled at 30% with a control statement increasing stirrer incrementally from 200 to 1200 rpm increasing by 25 rpm when PO2 drops below setpoint activated at 10-minute intervals and a fixed manual airflow of 4 litres of air per minute. The agitation was achieved by two conventional 6-flat bladed disc turbines and the airflow was sparged via a submerged sparger. pH was controlled at 7.2 with the automatic addition of 28% ammonium hydroxide. Fermentation substrate feed was applied to the fermenter from the start of inoculation, where it received a linear rate of 19.2 mL/hr to 103.1 mL/hr over 24 hours.

The linear feed was continued until the optical density reached 70 OD₆₀₀ and the culture was induced by the addition of 0.5 mM Isopropyl ß-D-1-thiogalactopyranoside (IPTG) and reduction of temperature to 25° C. The substrate feed rate was then switched to an event-based feeding method for the remainder of the production, adding 9 mL shot of feed when the dissolved oxygen rose above 30%. The fermentation was harvested once 20 hours had passed since induction. Fermentation broth was centrifuged at 8000 rcf at 4° C., 45 minutes and 751 g of cell pellet was frozen at −80° C. Cells solids were then resuspended in 50 mM potassium phosphate buffer pH 8.0 to a concentration of 30% solids. The slurry was then mechanically lysed using a french press cell disruptor at 1500 psi with 3 passes. Polyethyleneimine was added to the bulk homogenised lysate to a final concentration of 0.8% and agitated for 10 minutes before being centrifuged again at 8000 ×g for 30 minutes. The supernatant was retained, and the volume was concentrated by 50% using a 10 kDa MWCO PES filtration membrane. Retentate liquid was then dried under vacuum to create a lyophilised powder.

### Example 9. Carbon Isotope Characterization of Plant Derived UDCA

UDCA from two separate sources presumably derived from animal starting materials, were compared to UDCA derived from plant derived starting materials, made according to the methods of the current invention, for carbon and isotopic analysis. All analyses performed for elemental and isotopic analysis of carbon were conducted using isotope ratio mass spectrometers that utilize pneumatic type autosamplers, using two different quality control standards. The first standard is a pure chemical that is used to test the instrument linearity and define instrument response for the determination of elemental composition. Methionine (an amino acid) is typically the chemical standard used for this purpose. For each run, the effect of signal on isotopic measurement (linearity) is checked from 200 to 600 ug for carbon. The second standard is used to show measurement stability over the length of the run. This in-house standard is chosen to loosely resemble the matrix of the samples being analyzed. All in-house standards are calibrated periodically against international standards to verify accuracy. Within run isotopic precision for QC standards is 0.2 per mil for carbon. The test results are reported in Table 1.

TABLE 1

| Sample ID | Source | Weight (mg) | % C | δ¹³C v. VPDB |
|---|---|---|---|---|
| 320 A-4 | animal | 1.03 | 76.69 | −12.26 |
| 320 A-5 | animal | 1.06 | 75.47 | −12.21 |
| 320 A-6 | animal | 1.03 | 74.39 | −12.21 |
| 320 C-3 | animal | 0.99 | 78.12 | −12.26 |
| 320 C-4 | animal | 1.02 | 75.96 | −12.24 |
| 320 C-5 | animal | 1.02 | 75.51 | −12.19 |
| 320 A-7 | animal | 1.00 | 74.97 | −13.96 |
| 320 A-8 | animal | 1.00 | 74.80 | −13.94 |
| 320 A-9 | animal | 1.05 | 74.06 | −13.97 |
| 320 C-6 | animal | 1.01 | 75.29 | −14.01 |
| 320 C-7 | animal | 1.02 | 75.60 | −14.03 |
| 320 C-8 | animal | 1.00 | 76.09 | −13.99 |
| 320 A-10 | plant | 1.02 | 74.03 | −28.73 |
| 320 A-11 | plant | 1.03 | 74.38 | −28.73 |
| 320 C-9 | plant | 1.06 | 75.75 | −28.72 |
| 320 C-10 | plant | 1.02 | 75.15 | −28.71 |
| 320 C-11 | plant | 1.04 | 76.27 | −28.74 |
| 320 B-1 | plant | 1.03 | 79.43 | −28.74 |

### Example 10. Procedure for Purification of 3,7-DKCA by Way of 3,7-DKCA t-Butylamine Salt

To a 250 mL RBF was taken 3,7-DKCA (20 g) in EtOH (60 mL, 3 vol.) at RT. The mixture was stirred for 15 min to obtain a clear solution. To this solution was added tert-butylamine (TBA, 4.14 g, 1.1 equiv.) in EtOH (40 mL, 2 vol.) over a period of 0.5 h while stirring. A thick slurry was observed within 10 min. and another 20 mL EtOH was added. The suspension was stirred for 2 h at RT, the resulting solid was filtered, the wet cake was washed using cold EtOH (20 mL, 1 vol.) and the product was dried under vacuum to obtain the TBA salt of 3,7-DKCA (3,7-DKCA-TBA, 17.5 g) as an off-white solid.

The 3,7-DKCA-TBA obtained by the foregoing process (34.8 g) was suspended in toluene (174 mL, 5 vol.). The resulting slurry was stirred at 45° C. for 0.5 h and treated with EtOH (522 mL, 15 vol.) at 45° C. The resulting mixture stirred for 20 min to obtain a clear solution. The solvent was evaporated under reduced pressure until ˜7 volumes remained (solid precipitation was observed). Additional EtOH (522 mL, 15 vol.) was added and the solvent was evaporated under reduced pressure until ˜5 volumes remained. The slurry was treated with additional EtOH (174 mL, 5 volumes), stirred at RT for 1 h and the solid was filtered. The wet cake was washed using EtOH (1 vol.) and the solid was dried under vacuum to obtain purified 3,7-DKCA-TBA (22 g) as a white solid.

Melting point=144° C.; Purity by CAD HPLC (area-%)=98% (5α-impurity=not detected); ¹H-NMR (400 MHz, CDCl₃): δ 6.62 (bs, 3H), 2.87 (dd, J=12.4 & 5.6 Hz, 1H), 2.49 (t, J=11.4 Hz, 1H), 0.95-2.40 (m, 27H), 1.31 (s, 9H), 0.93 (d, J=6.4 Hz, 3H), 0.68 (s, 3H).

### Conversion of DKCA-TBA Salt to DKCA

DKCA-TBA salt (20 g) was suspended in water (100 mL). Ethyl acetate (100 mL) was added, followed by 6N HCl (7 mL), leading to a two-phase mixture without any solids. The phases were separated and the organic phase was washed with 1N HCl (20 mL) and then with water (40 mL). The ethyl acetate phase was then concentrated under vacuum to dryness to give a white solid (16 g, 95% yield).

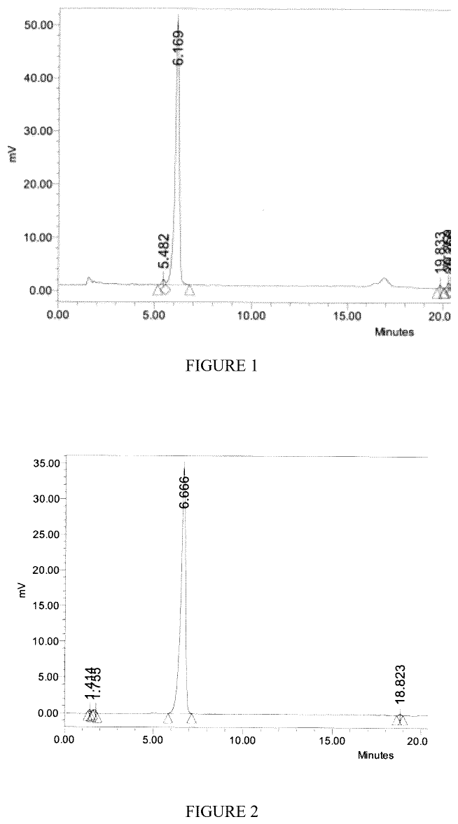

Using validated HPLC methods for measuring the presence of 5α-impurities, the 3,7-DKCA starting material and the final product produced in this Example 10 were subjected to HPLC analysis. The results are depicted in FIG. 1 and FIG. 2, respectively. The 5α-impurity of 3,7-DKCA (RRT 0.88) had an area % of 1.3. The 5α-impurity was not detectable in the final 3,7-DKCA.

### Example 11. Crystalline Salts of 3,7-DKCA

Tert-butylamine, ethylenediamine, and diisopropylamine salts of 3,7-DKCA were crystallized, characterized and scaled up. All three salts showed significant increases in purity, including considerable rejection of the impurity markers of interest. The ethylenediamine salt was observed to be quite polymorphic, with six different forms observed throughout the work. The diisopropylamine salt demonstrated high crystallinity and satisfactory purity results, and considerable mass loss by thermogravimetric analysis (TGA) coincident with an endotherm that had an onset of approximately 86° C. The tert-butylamine salt had high crystallinity, thermal behavior (melting onset at 143.7° C.), and ability to purge impurities, including markers of interest.

Select properties of the products obtained are presented in Table 2.

TABLE 2
Summary of the top results.

| Description | Results | | | |
|---|---|---|---|---|
| Sample | tert- | | Ethylene- | Diisopropyl- |
| | Butylamine | | diamine | amine salt |
| | salt | | salt | |
| XRPD pattern | 9-A | | 6-D | 10-A |
| DSC onsets (° C.) | 143.70 | | 192.58 | 83.48, 120.70 |
| TGA mass loss (wt. %) | 13.15 | | 5.78 | 5.44 |
| Stoichiometry by ¹H NMR | 1.00:1.06 | | 1.00:0.75 | 1.00:0.88 |
| (FF:CI) | | | | |
| KF water content (wt. %) | <0.02 | | 0.86 | 2.40 |
| HPLC chemical purity | 97.63 | 99.29 | 98.88 | 97.33 |

| (% a/a) | | | | |
|---|---|---|---|---|
| DVS mass change | | 10.89 | 19.60 | 2.53 |
| 5-95% RH (wt. %) | | | | |
| Stability at 40° C. and | | 9-A | 6-D | 10-A |
| 75% RH | | | | |
| One-week form | EtOAc | 9-A | 6-D | 10-A |
| stability in | 2-MeTHF | 9-A | 6-D + 6-B | 10-A |
| solvents | | | (trace) | |
| | ACN:water | 9-A | 6-F | — |
| | (9:1 vol.) | | | |

Note.
CI, counter ion; N/A, not applicable. Hyphen indicates no data were collected.

### Pattern 9-A Scale-Up and XRPD Characterization

Pattern 9-A (tert-Butylamine salt) was scaled up to carry out further characterization. A yield of 123.23 mg (40.0% w/w) with a purity of 99.29% a/a was obtained. The crystallization process was as follows:
1. Weighed 351.4 mg of 3,7-DKCA into a 20 mL vial
2. Added 4 mL EtOH to the vial
3. Stirred at RT until solid dissolved
4. Weighed 67.1 mg (1.1 equivalents (eq.)) of tert-butylamine into a new 20 mL vial
5. Added 4 mL of EtOH to the vial containing the counter ion
6. Added the counter ion solution to the freeform solution dropwise at RT while stirring at 500 rpm
7. Seeded with 5 mg of Pattern 9-A
8. Heated the solution to 45° C. and stirred for 4 h
9. Cooled down to RT while stirring
10. Filtered and washed with 3 vol. of EtOH
11. Dried in an oven at 50° C. and −29 inHg overnight
   An XRPD peak listing for scaled-up Pattern 9-A is given in Table 3. An XRPD pattern is depicted in FIG. 3.

TABLE 3
XRPD peak list for scaled-up Pattern 9-A.

| Angle | d-spacing | Relative |
|---|---|---|
| (°2θ) | (Å) | intensity |
| 4.83 | 18.28 | 100 |
| 8.77 | 10.07 | 16 |
| 9.68 | 9.13 | 2 |
| 9.90 | 8.93 | 24 |
| 11.67 | 7.58 | 1 |
| 14.08 | 6.29 | 1 |
| 13.35 | 6.63 | 59 |
| 14.22 | 6.23 | 9 |
| 14.46 | 6.12 | 10 |
| 15.56 | 5.69 | 10 |
| 16.03 | 5.52 | 4 |
| 16.49 | 5.37 | 36 |
| 16.65 | 5.32 | 21 |
| 17.51 | 5.06 | 12 |
| 17.76 | 4.99 | 3 |
| 18.92 | 4.69 | 2 |
| 19.30 | 4.60 | 2 |
| 19.73 | 4.50 | 7 |
| 20.10 | 4.41 | 7 |
| 20.54 | 4.32 | 8 |
| 20.95 | 4.24 | 6 |
| 22.05 | 4.03 | 12 |
| 23.53 | 3.78 | 5 |
| 24.00 | 3.70 | 3 |
| 24.18 | 3.68 | 2 |
| 24.75 | 3.59 | 5 |
| 25.28 | 3.52 | 1 |
| 25.75 | 3.46 | 1 |
| 27.26 | 3.27 | 2 |
| 27.52 | 3.24 | 2 |
| 27.71 | 3.22 | 4 |
| 28.27 | 3.15 | 1 |
| 28.70 | 3.11 | 1 |
| 28.88 | 3.09 | 1 |
| 29.12 | 3.06 | 1 |
| 29.93 | 2.98 | 2 |
| 30.40 | 2.94 | 3 |
| 31.97 | 2.80 | 2 |
| 32.14 | 2.78 | 1 |
| 33.66 | 2.66 | 1 |
| 34.20 | 2.62 | 1 |
| 34.91 | 2.57 | 1 |
| 36.43 | 2.46 | 1 |
| 36.74 | 2.44 | 1 |

Note.
The peak cut off used was <1% relative intensity.

### Pattern 6-D Scale-Up and XRPD Characterization

Pattern 6-D Ethylenediamine was scaled up to carry out further characterization. A yield of 207 mg (65.7% w/w) with a purity of 98.88% a/a was obtained. The crystallization process was as follows:
1. Weighed 357.2 mg of 3,7-DKCA (L1FL120004-2-1) into a 20 mL vial
2. Added 2 mL EtOH to the vial
3. Stirred at RT until solid dissolved
4. Weighed 56.5 mg (1.1 eq.) of ethylenediamine into a new 20 mL vial
5. Added 2 mL of EtOH to the vial containing the counter ion
6. Added the freeform solution to the counter ion solution dropwise at RT while stirring at 500 rpm
7. Heated the solution to 45° C. and stirred for 2 h
8. Evaporated the solvent under nitrogen flow at 45° C.
9. Dried in an oven at 50° C. and −29 inHg for 2 h
10. Added 3 vol. IPA:water (9:1 vol.) to the vial and stirred at RT
11. Heated to 50° C.
12. Cooled down to 5° C. over 12 h
13. Filtered and washed with 3 vol. of IPA:water (9:1 vol.)
14. Dried in an oven at 50° C. and −29 inHg for 3 h
   An XRPD peak list for Pattern 6-D is provided in Table 4. An XRPD pattern is depicted in FIG. 4.

TABLE 4
XRPD peak list for scaled-up Pattern 6-D.

| Angle | d-Spacing | Relative |
|---|---|---|
| (°2θ) | (Å) | intensity (%) |
| 5.81 | 15.19 | 27 |
| 8.69 | 10.17 | 3 |
| 9.95 | 8.88 | 67 |
| 10.92 | 8.10 | 22 |
| 11.60 | 7.62 | 3 |
| 13.08 | 6.76 | 16 |
| 13.78 | 6.42 | 5 |
| 14.59 | 6.07 | 94 |
| 15.19 | 5.83 | 10 |
| 15.38 | 5.76 | 22 |
| 16.03 | 5.52 | 100 |
| 16.51 | 5.37 | 8 |
| 17.40 | 5.09 | 40 |
| 17.77 | 4.99 | 10 |
| 18.11 | 4.89 | 5 |
| 18.89 | 4.69 | 57 |
| 19.21 | 4.62 | 29 |
| 19.94 | 4.45 | 10 |
| 20.27 | 4.38 | 3 |
| 21.04 | 4.22 | 3 |
| 21.32 | 4.16 | 4 |
| 22.10 | 4.02 | 9 |
| 22.27 | 3.99 | 8 |
| 23.45 | 3.79 | 18 |
| 24.04 | 3.70 | 4 |
| 24.24 | 3.67 | 4 |
| 24.90 | 3.57 | 1 |
| 25.11 | 3.54 | 2 |
| 25.60 | 3.48 | 1 |
| 26.00 | 3.42 | 2 |
| 26.23 | 3.39 | 7 |
| 26.82 | 3.32 | 1 |
| 27.42 | 3.25 | 6 |
| 28.10 | 3.17 | 2 |
| 28.33 | 3.15 | 3 |
| 28.82 | 3.10 | 2 |
| 29.69 | 3.01 | 2 |
| 29.88 | 2.99 | 2 |
| 30.24 | 2.95 | 2 |
| 31.57 | 2.83 | 1 |
| 32.36 | 2.76 | 1 |
| 32.59 | 2.75 | 1 |
| 32.87 | 2.72 | 1 |
| 33.35 | 2.68 | 4 |
| 34.73 | 2.58 | 1 |
| 35.18 | 2.55 | 1 |
| 35.54 | 2.52 | 1 |
| 36.07 | 2.49 | 1 |
| 36.70 | 2.45 | 1 |
| 37.24 | 2.41 | 1 |
| 37.52 | 2.39 | 2 |
| 37.83 | 2.38 | 6 |
| 38.22 | 2.35 | 3 |
| 39.42 | 2.28 | 1 |
| 39.80 | 2.26 | 1 |

Note.
The peak cut off used was <1% relative intensity.

### Pattern 10-A Scale-Up and XRDP Characterization

Pattern 10-A (diisopropylamine salt) was scaled up to carry out further characterization. A yield of 175.8 mg (57.1% w/w) with a purity of 97.33% a/a was obtained. The crystallization process was as follows:
1. Weighed 348.2 mg of 3,7-DKCA (L1FL120004-2-1) into a 20 mL vial
2. Added 2 mL EtOH to the vial
3. Stirred at RT until solid dissolved
4. Weighed 1.1 eq. of diisopropylamine into a new 20 mL vial
5. Added 1 mL of EtOH to the vial containing the counter ion
6. Added the freeform solution to the counter ion solution at RT while stirring at 500 rpm
7. Heated the solution to 45° C. and stirred for 2 h
8. Evaporated the solvent under nitrogen flow at 45° C.
9. Dried in an oven at 50° C. and −29 inHg for 3 h
10. Added 10 vol. MIBK to the vial and stirred at 45° C. for 1 h
11. Cooled down to 25° C.
12. Added 3 vol. heptane dropwise over 15 min
13. Seeded with Pattern 10-A (L1FL120004-7-33)
14. Stirred at RT and 500 rpm over the weekend
15. Filtered and washed with 3 vol. of MIBK:heptane (3:1 vol.)
16. Dried in an oven at 50° C. and −29 inHg for 3 h
   An XRPD peak list for Pattern 10-A is provided in Table 5. An XRPD pattern is depicted in FIG. 5.

TABLE 5
XRPD peak list for scaled-up Pattern 10-A.

| Angle | d-Spacing | Relative intensity |
|---|---|---|
| (°2θ) | (Å) | (%) |
| 5.85 | 15.09 | 43 |
| 6.29 | 14.03 | 4 |
| 9.05 | 9.76 | 41 |
| 11.46 | 7.71 | 57 |
| 11.88 | 7.44 | 21 |
| 12.58 | 7.03 | 26 |
| 13.07 | 6.77 | 63 |
| 13.40 | 6.60 | 3 |
| 13.55 | 6.53 | 5 |
| 14.17 | 6.25 | 16 |
| 14.61 | 6.06 | 100 |
| 14.82 | 5.97 | 4 |
| 16.09 | 5.51 | 36 |
| 17.03 | 5.20 | 2 |
| 17.37 | 5.10 | 3 |
| 17.65 | 5.02 | 8 |
| 18.13 | 4.89 | 68 |
| 18.47 | 4.80 | 32 |
| 18.89 | 4.69 | 3 |
| 19.79 | 4.48 | 9 |
| 20.00 | 4.44 | 10 |
| 20.49 | 4.33 | 15 |
| 21.48 | 4.13 | 2 |
| 23.08 | 3.85 | 23 |
| 23.31 | 3.81 | 10 |
| 23.50 | 3.78 | 5 |
| 23.86 | 3.73 | 4 |
| 24.13 | 3.69 | 12 |
| 24.75 | 3.59 | 2 |
| 25.27 | 3.52 | 2 |
| 25.78 | 3.45 | 6 |
| 26.03 | 3.42 | 6 |
| 26.17 | 3.40 | 6 |
| 26.66 | 3.34 | 1 |
| 26.94 | 3.31 | 1 |
| 27.10 | 3.29 | 2 |
| 27.26 | 3.27 | 3 |
| 27.80 | 3.21 | 2 |
| 28.19 | 3.16 | 4 |
| 28.65 | 3.11 | 2 |
| 29.23 | 3.05 | 3 |
| 29.41 | 3.03 | 4 |
| 30.21 | 2.96 | 2 |
| 30.66 | 2.91 | 3 |
| 31.02 | 2.88 | 3 |
| 31.48 | 2.84 | 1 |
| 31.82 | 2.81 | 2 |
| 32.46 | 2.76 | 4 |
| 32.74 | 2.73 | 3 |
| 34.50 | 2.60 | 1 |
| 34.78 | 2.58 | 2 |
| 34.89 | 2.57 | 2 |
| 35.06 | 2.56 | 3 |
| 35.28 | 2.54 | 2 |
| 37.44 | 2.40 | 2 |

Note.
The peak cut off used was <1% relative intensity.

### Example 12. Purity Characterization

The foregoing examples and general description have illustrated how to obtain UDCA of remarkable purity, particularly UDCA derived from non-animal sources. Three of the major impurities implicated in the manufacture of UDCA, particularly UDCA from non-animal sources, are controlled as described below:
5-alpha impurities are controlled by hydrogenation with pyridine solvents, and by the formation and crystallization of crystalline salts of DKCA to give undetectable levels of the 5-alpha isomer of DKCA and other 5-alpha impurities;
3-beta impurities are controlled by the use of 3-alpha HSDH (a/k/a ketoreductase) to reduce the 3-ketone, such that no 3-beta impurities are formed or detected in the intermediates or final product UDCA;
7-alpha impurities are controlled by use of 7-beta-HSDH (a/k/a ketoreductase) to reduce the 7-ketone, such that no 7-alpha impurities are formed or detected in the intermediates or final product UDCA;

To confirm the lack of these impurities, various analyses were undertaken of the final UDCA produced by the methods of the invention and intermediates thereof. The results are summarized as follows:

FIG. 6 is an HPLC chromatogram of tert-butylamine salt of 3,7-DKCA produced substantially according to the 3-picoline solvent hydrogenation and tert-butylamine crystallization methods described herein. The dominant peak is tert-butylamine salt of 3,7-DKCA. The 5-alpha impurity of 3,7-DKCA or the tert-butylamine salt of 3,7-DKCA is undetectable.

Table 6 reports purity testing of UDCA obtained by reducing the 3- and 7-keto groups on 3,7-DKCA using the keto-reductases described herein. The 3-picoline solvent hydrogenation and tert-butylamine crystallization methods described herein were also employed. No 3-beta impurity was detected, and the levels of 5-alpha and 7-alpha impurities are very low.

TABLE 6

| | | |
|---|---|---|
| 225RKS-K-117 | Impurity | Percentage |

| | |
|---|---|
| 3-beta | ND (<0.03%) |
| 5-alpha | 0.07% |
| 7-alpha | 0.03% |

ND = none detected

### REFERENCES CITED

Zhang, Y., Werling, U., Ederlmann, W. (2014). Seamless Ligation Cloning Extract (SLiCE) Cloning Method. Methods in Molecular Biology 1116, 235-244.

SEQUENCE LISTING

A sequence listing is filed herewith.
<110> Sandhill One
<120> TBC
<130> TBC
<160> 31
<170> PatentIn version 3.5
<210> 1
<211> 2491
<212> DNA
<213> Artificial sequence
<220>
<223> synthetic DNA
<400> 1

| | |
|---|---|
| taaactggct gacggaattt atgcctcttc cgaccatcaa gcattttatc cgtactcctg | 60 |
| atgatgcatg gttactcacc actgcgatcc ccgggaaaac agcattccag gtattagaag | 120 |
| aatatcctga ttcaggtgaa aatattgttg atgcgctggc agtgttcctg cgccggttgc | 180 |
| attcgattcc tgtttgtaat tgtcctttta acagcgatcg cgtatttcgt ctcgctcagg | 240 |
| cgcaatcacg aatgaataac ggtttggttg atgcgagtga ttttgatgac gagcgtaatg | 300 |
| gctggcctgt tgaacaagtc tggaaagaaa tgcataaact tttgccattc tcaccggatt | 360 |
| cagtcgtcac tcatggtgat ttctcacttg ataaccttat ttttgacgag gggaaattaa | 420 |
| taggttgtat tgatgttgga cgagtcggaa tcgcagaccg ataccaggat cttgccatcc | 480 |
| tatggaactg cctcggtgag ttttctcctt cattacagaa acggcttttt caaaaatatg | 540 |
| gtattgataa tcctgatatg aataaattgc agtttcattt gatgctcgat gagtttttct | 600 |
| aagaattaat tcatgagcgg atacatattt gaatgtattt agaaaaataa acaaataggg | 660 |
| gttccgcgca catttccccg aaaagtgcca cctgaaattg taaacgttaa tattttgtta | 720 |
| aaattcgcgt taaatttttg ttaaatcagc tcatttttta accaataggc cgaaatcggc | 780 |
| aaaatccctt ataaatcaaa agaatagacc gagatagggt tgagtgttgt tccagtttgg | 840 |
| aacaagagtc cactattaaa gaacgtggac tccaacgtca aagggcgaaa aaccgtctat | 900 |
| cagggcgatg gcccactacg tgaaccatca ccctaatcaa gttttttggg gtcgaggtgc | 960 |
| cgtaaagcac taaatcggaa ccctaaaggg agcccccgat ttagagcttg acggggaaag | 1020 |
| ccggcgaacg tggcgagaaa ggaagggaag aaagcgaaag gagcgggcgc tagggcgctg | 1080 |
| gcaagtgtag cggtcacgct gcgcgtaacc accacacccg cegcgcttaa tgcgccgcta | 1140 |
| cagggcgcgt cccattcgcc aatccggata tagttcctcc tttcagcaaa aaacccctca | 1200 |
| agacccgttt agaggcccca aggggttatg ctagttattg ctcagcggtg gcagcagcca | 1260 |
| actcagcttc ctttcgggct ttgttagcag ccggatctca gtggtggtgg tggtggtgct | 1320 |
| cgagtgcggc cgcaagcttg tcgacggagc tcgaattcgg atccgcgacc catttgctgt | 1380 |
| ccaccagtca tgctagccat atggctgccg cgcggcacca ggccgctgct gtgatgatga | 1440 |
| tgatgatggc tgctgcccat ggtatatctc cttcttaaag ttaaacaaaa ttatttctag | 1500 |
| aggggaattg ttatccgctc acaattcccc tatagtgagt cgtattaatt tcgcgggatc | 1560 |
| gagatctcga tcctctacgc cggacgcatc gtggccggca tcaccggcgc cacaggtgcg | 1620 |
| gttgctggcg cctatatcgc cgacatcacc gatggggaag atcgggctcg ccacttcggg | 1680 |
| ctcatgagcg cttgtttcgg cgtgggtatg gtggcaggcc ccgtggccgg gggactgttg | 1740 |
| ggcgccatct ccttgcatgc accattcctt gcggcggcgg tgctcaacgg cctcaaccta | 1800 |
| ctactgggct gcttcctaat gcaggagtcg cataagggag agcgtcgaga tcccggacac | 1860 |
| catcgaatgg cgcaaaacct ttcgcggtat ggcatgatag cgcccggaag agagtcaatt | 1920 |
| cagggtggtg aatgtgaaac cagtaacgtt atacgatgtc gcagagtatg ccggtgtctc | 1980 |
| ttatcagacc gtttcccgcg tggtgaacca ggccagccac gtttctgcga aaacgcggga | 2040 |
| aaaagtggaa gcggcgatgg cggagctgaa ttacattccc aaccgcgtgg cacaacaact | 2100 |
| ggcgggcaaa cagtcgttgc tgattggcgt tgccacctcc agtctggccc tgcacgcgcc | 2160 |
| gtcgcaaatt gtcgcggcga ttaaatctcg cgccgatcaa ctgggtgcca gcgtggtggt | 2220 |
| gtcgatggta gaacgaagcg gcgtcgaagc ctgtaaagcg gcggtgcaca atcttctcgc | 2280 |
| gcaacgcgtc agtgggctga tcattaacta tccgctggat gaccaggatg ccattgctgt | 2340 |
| ggaagctgcc tgcactaatg ttccggcgtt atttcttgat gtctctgacc agacacccat | 2400 |
| caacagtatt attttctccc atgaagacgg tacgcgactg ggcgtggagc atctggtcgc | 2460 |
| attgggtcac cagcaaatcg cgctgttagc g | 2491 |
| <210> 2 | |
| <211> 74 | |
| <212> DNA | |
| <213> Artificial sequence | |
| <220> | |
| <223> primer | |
| <400> 2 | |
| acatggcaaa ggtagcgttg ccaatgatgt tacagatgag atggtcagac aaactggct | 60 |
| gacggaattt atgc | 74 |
| <210> 3 | |
| <211> 20 | |
| <212> DNA | |
| <213> Artificial sequence | |
| <220> | |
| <223> primer | |
| <400> 3 | |
| cgctaacagc gcgatttgct | 20 |
| <210> 4 | |
| <211> 2877 | |
| <212> DNA | |
| <213> Artificial sequence | |
| <220> | |
| <223> synthetic DNA | |
| <210> 4 | |
| ggcccattaa gttctgtctc ggcgcgtctg cgtctggctg gctggcataa atatctcact | 60 |
| cgcaatcaaa ttcagccgat agcggaacgg gaaggcgact ggagtgccat gtccggtttt | 120 |
| caacaaacca tgcaaatgct gaatgagggc atcgttccca ctgcgatgct ggttgccaac | 180 |
| gatcagatgg cgctgggcgc aatgcgcgcc attaccgagt ccgggctgcg cgttggtgcg | 240 |
| gatatctcgg tagtgggata cgacgatacc gaagacagct catgttatat cccgccgtta | 300 |
| accaccatca aacaggattt tcgcctgctg gggcaaacca gcgtggaccg cttgctgcaa | 360 |
| ctctctcagg gccaggcggt gaagggcaat cagctgttgc ccgtctcact ggtgaaaaga | 420 |
| aaaaccaccc tggcgcccaa tacgcaaacc gcctctcccc gcgcgttggc cgattcatta | 480 |
| atgcagctgg cacgacaggt ttcccgactg gaaagcgggc agtgagcgca acgcaattaa | 540 |
| tgtaagttag ctcactcatt aggcaccggg atctcgaccg atgcccttga gagccttcaa | 600 |
| cccagtcagc tccttccggt gggcgcgggg catgactatc gtcgccgcac ttatgactgt | 660 |
| cttctttatc atgcaactcg taggacaggt gccggcagcg ctctgggtca ttttcggcga | 720 |
| ggaccgcttt cgctggagcg cgacgatgat cggcctgtcg cttgccatgc gagacccttg | 780 |
| cacgccctcg ctcaagcctt cgtcactggt cccgccacca aacgttggtc tcggccgcag | 840 |
| gccattatcg ccggcatggc ggccccacgg gtgcgcatga tcgtgctcct gtcgttgagg | 900 |
| acccggctag gctggcgggg ttgccttact ggttagcaga atgaatcacc gatacgcgag | 960 |
| cgaacgtgaa gcgactgctg ctgcaaaacg tctgcgacct gagcaacaac atgaatggtc | 1020 |
| ttcggtttcc gtgtttcgta aagtctggaa acgcggaagt cagcgccctg caccattatg | 1080 |
| ttccggatct gcatcgcagg atgctgctgg ctaccctgtg gaacacctac atctgtatta | 1140 |
| acgaagcgct ggcattgacc ctgagtgatt tttctctggt cccgccgcat ccataccgcc | 1200 |
| agttgtttac cctcacaacg ttccagtaac cgggcatgtt catcatcagt aacccgtatc | 1260 |
| gtgagcatcc tctctcgttt catcggtatc attaccccca tgaacagaaa tcccccttac | 1320 |
| acggaggcat cagtgaccaa acaggaaaaa accgccctta acatggcccg ctttatcaga | 1380 |
| agccagacat taacgcttct ggagaaactc aacgagctgg acgcggatga acaggcagac | 1440 |
| atctgtgaat cgcttcacga ccacgctgat gagctttacc gcagctgcct cgcgcgtttc | 1500 |
| ggtgatgacg gtgaaaacct ctgacacatg cagctcccgg agacggtcac agcttgtctg | 1560 |
| taagcggatg ccgggagcag acaagcccgt cagggcgcgt cagcgggtgt tggcgggtgt | 1620 |
| cggggcgcag ccatgaccca gtcacgtagc gatagcggag tgtatactgg cttaactatg | 1680 |
| cggcatcaga gcagattgta ctgagagtgc accatatatg cggtgtgaaa taccgcacag | 1740 |
| atgcgtaagg agaaaatacc gcatcaggcg ctcttccgct tectcgctca ctgactcgct | 1800 |
| gcgctcggtc gttcggctgc ggcgagcggt atcagctcac tcaaaggcgg taatacggtt | 1860 |
| atccacagaa tcaggggata acgcaggaaa gaacatgtga gcaaaaggcc agcaaaaggc | 1920 |
| caggaaccgt aaaaaggccg cgttgctggc gtttttccat aggctccgcc cccctgacga | 1980 |
| gcatcacaaa aatcgacgct caagtcagag gtggcgaaac ccgacaggac tataaagata | 2040 |
| ccaggcgttt ccccctggaa gctccctcgt gcgctctcct gttccgaccc tgccgcttac | 2100 |
| cggatacctg tccgcctttc tcccttcggg aagcgtggcg ctttctcata gctcacgctg | 2160 |
| taggtatctc agttcggtgt aggtcgttcg ctccaagctg ggctgtgtgc acgaaccccc | 2220 |
| cgttcagccc gaccgctgcg ccttatccgg taactatcgt cttgagtcca acccggtaag | 2280 |
| acacgactta tcgccactgg cagcagccac tggtaacagg attagcagag cgaggtatgt | 2340 |
| aggcggtgct acagagttct tgaagtggtg gcctaactac ggctacacta gaaggacagt | 2400 |
| atttggtatc tgcgctctgc tgaagccagt taccttcgga aaaagagttg gtagctcttg | 2460 |
| atccggcaaa caaaccaccg ctggtagcgg tggttttttt gtttgcaagc agcagattac | 2520 |
| gcgcagaaaa aaaggatctc aagaagatcc tttgatcttt tctacggggt ctgacgctca | 2580 |
| gtggaacgaa aactcacgtt aagggatttt ggtcatgaac aataaaactg tctgcttaca | 2640 |
| taaacagtaa tacaaggggt gttatgagcc atattcaacg ggaaacgtct tgctctaggc | 2700 |
| cgcgattaaa ttccaacatg gatgctgatt tatatgggta taaatgggct cgcgataatg | 2760 |
| tcgggcaatc aggtgcgaca atctatcgat tgtatgggaa gcccgatgcg ccagagttgt | 2820 |
| ttctgaaaca tggcaaaggt agcgttgcca atgatgttac agatgagatg gtcagac | 2877 |
| <210> 5 | |
| <211> 71 | |
| <212> DNA | |
| <213> Artificial sequence | |
| <220> | |
| <223> primer | |
| <400> 5 | |
| gcgtggagca tctggtcgca ttgggtcacc agcaaatcgc gctgttagcg ggcccattaa | 60 |
| gttctgtctc g | 71 |
| <210> 6 | |
| <211> 24 | |
| <212> DNA | |
| <213> Artificial sequence | |
| <220> | |
| <223> primer | |
| <400> 6 | |
| gtctgaccat ctcatctgta acat | 24 |
| <210> 7 | |
| <211> 20 | |
| <212> DNA | |
| <213> Artificial sequence | |
| <220> | |
| <223> primer | |
| <400> 7 | |
| catcggtgat gtcggcgata | 20 |
| <210> 8 | |
| <211> 67 | |
| <212> DNA | |
| <213> Artificial sequence | |
| <220> | |
| <223> primer | |
| <400> 8 | |
| tacggggtct gacgctcagt ggaacgaaaa ctcacgttaa gggattttgg cgcggaaccc | 60 |
| ctatttg | 67 |
| <210> 9 | |
| <211> 26 | |
| <212> DNA | |
| <213> Artificial sequence | |
| <220> | |
| <223> primer | |
| <400> 9 | |
| cactagtgaa tcggccaacg cgcggg | 26 |
| <210> 10 | |
| <211> 25 | |
| <212> DNA | |
| <213> Artificial sequence | |
| <220> | |
| <223> primer | |
| <400> 10 | |
| gctgagcaat aactagcata acccc | 25 |
| <210> 11 | |
| <211> 37 | |
| <212> DNA | |
| <213> Artificial sequence | |
| <220> | |
| <223> primer | |
| <400> 11 | |
| ggtatatctc cttcttaaag ttaaacaaaa ttatttc | 37 |
| <210> 12 | |
| <211> 774 | |
| <212> DNA | |
| <213> Artificial sequence | |
| <220> | |
| <223> synthetic DNA | |
| <400> 12 | |
| atgtctatca tcgttatctc tggctgtgcg acgggtattg gcgcagctac tcgtaaagtc | 60 |
| ctggaagcag cgggccacca gatcgttggc attgacattc gtgacgccga ggttatcgct | 120 |
| gacctgtcta ccgcagaggg ccgtaaacag gcgattgctg atgttctggc taagtgttct | 180 |
| aaaggcatgg atggtctggt tctgtgtgcg ggtctgggtc cgcagaccaa agttctgggt | 240 |
| aacgtagtga gcgttaacta cttcggcgca accgaactga tggatgcttt cctgcctgca | 300 |
| ctgaaaaaag gccatcaacc ggccgcggta gtgattagca gcgttgcttc tgcgcacctg | 360 |
| gcgttcgata aaaacccact ggcgctggca ctggaagctg gcgaagaagc aaaagcccgt | 420 |
| gcaattgtag aacacgctgg tgaacagggt ggtaacctgg cgtacgctgg ctctaagaat | 480 |
| gctctgaccg ttgctgttcg taaacgtgct gctgcctggg gtgaagccgg tgttcgtctg | 540 |
| aacactatcg cgccgggtgc tactgaaacg ccactgctgc aagcgggcct gcaggatcca | 600 |
| cgttacggcg aatccattgc taaattcgtt cctccgatgg gccgtcgtgc tgaaccatct | 660 |
| gaaatggcta gcgttatcgc attcctgatg tctccggctg catcttatgt tcacggtgcc | 720 |
| cagatcgtca tcgatggtgg catcgatgca gtcatgcgtc ctactcaatt ctga | 774 |

<210> 13
<211> 74
<212> DNA

| | |
|---|---|
| <213> Artificial sequence | |
| <220> | |
| <223> primer | |
| <400> 13 | |
| caattcccct ctagaaataa ttttgtttaa ctttaagaag gagatatacc atgtctatca | 60 |
| tcgttatctc tggc | 74 |
| <210> 14 | |
| <211> 75 | |
| <212> DNA | |
| <213> Artificial sequence | |
| <220> | |
| <223> primer | |
| <400> 14 | |
| ctcaagaccc gtttagaggc cccaaggggt tatgctagtt attgctcagc tcagaattga | 60 |
| gtaggacgca tgact | 75 |
| <210> 15 | |
| <211> 20 | |
| <212> DNA | |
| <213> Artificial sequence | |
| <220> | |
| <223> primer | |
| <400> 15 | |
| cccattcgcc aatccggata | 20 |
| <210> 16 | |
| <211> 6593 | |
| <212> DNA | |
| <213> Artificial sequence | |
| <220> | |
| <223> plasmid | |
| <400> 16 | |
| taatacgact cactataggg gaattgtgag cggataacaa ttcccctcta gaaaaaagct | 60 |
| tataattatc cttacatttc ccgtgagtgc gcccagatag ggtgttaagt caagtagttt | 120 |
| aaggtactac tctgtaagat aacacagaaa acagccaacc taaccgaaaa gcgaaagctg | 180 |
| atacgggaac agagcacggt tggaaagcga tgagttacct aaagacaatc gggtacgact | 240 |
| gagtcgcaat gttaatcaga tataaggtat aagttgtgtt tactgaacgc aagtttctaa | 300 |
| tttcggttaa atgtcgatag aggaaagtgt ctgaaacctc tagtacaaag aaaggtaagt | 360 |
| tacgtcacgg gacttatctg ttatcaccac atttgtacaa tctgtaggag aacctatggg | 420 |
| aacgaaacga aagcgatgcc gagaatctga atttaccaag acttaacact aactggggat | 480 |
| accctaaaca agaatgccta atagaaagga ggaaaaaggc tatagcacta gagcttgaaa | 540 |
| atcttgcaag ggtacggagt actcgtagta gtctgagaag ggtaacgccc tttacatggc | 600 |
| aaaggggtac agttattgtg tactaaaatt aaaaattgat tagggaggaa aacctcaaaa | 660 |
| tgaaaccaac aatggcaatt ttagaaagaa tcagtaaaaa ttcacaagaa aatatagacg | 720 |
| aagtttttac aagactttat cgttatcttt tacgtccaga tatttattac gtggcgacgc | 780 |
| gttacagcaa gcgaaccgga attgccagct ggggcgccct ctggtaaggt tgggaagccc | 840 |
| tgcaaagtaa actggatggc tttcttgccg ccaaggatct gatggcgcag gggatcaaga | 900 |
| tctgatcaag agacaggatg aggatcgttt cgcatgatcg agcaggacgg tttacatgcg | 960 |
| ggctcgcctg ctgcctgggt tgaacgctta tttggttacg attgggcgca gcaaaccatt | 1020 |
| gggtgttcag acgcggcggt ctttcgtttg tcggctcaag gtcgtcctgt gctgttcgtt | 1080 |
| aaaacagatt taagcggggc gttgaacgag ttgcaagatg aagcggcacg tctcagctgg | 1140 |
| cttgcgacta caggagtacc gtgtgccgcc gtactggatg tcgtaaccga ggccgggcgt | 1200 |
| gattggttgt tgttaggtga ggtacccgga caagacctgt taagctccca tttggccccg | 1260 |
| geggaaaagg ttagcattat ggcggacgcc atgcgtcgct tgcacaccct ggaccccgca | 1320 |
| acgtgtccgt ttgatcatca ggcaaagcac cgtattgaac gtgcgcgcac acgtatggaa | 1380 |
| gcggggctgg tagaccaaga cgacctcgat gaggaacacc aaggcctggc cccggcagag | 1440 |
| ttatttgcgc gcttgaaagc ccgtatgcct gatggtgaag acctggtggt cacacacggg | 1500 |
| gacgcatgtc ttccaaacat tatggtcgag aacggtcgtt tctcgggctt tattgattgc | 1560 |
| ggacgccttg gcgtcgccga tcgttaccaa gatatcgccc ttgcaacgcg cgacatcgcg | 1620 |
| gaagaactgg gtggtgagtg ggcagatcgc tttctggtac tgtatgggat tgcggccccg | 1680 |
| gactcccaac gtattgcttt ctaccgtctg ctcgatgaat tcttctaata aacttgcacg | 1740 |
| cgttgggaaa tggcaatgat agcgaaacaa cgtaaaactc ttgttgtatg ctttcattgt | 1800 |
| catcgtcacg tgattcataa acacaagtga atgtcgacag tgaattttta cgaacgaaca | 1860 |
| ataacagagc cgtatactcc gagaggggta cgtacggttc ccgaagaggg tggtgcaaac | 1920 |
| cagtcacagt aatgtgaaca aggcggtacc tccctacttc accatatcat tttctgcagc | 1980 |
| cccctagaaa taattttgtt taactttaag aaggagatat acatatatgg ctagatcgtc | 2040 |
| cattccgaca gcatcgccag tcactatggc gtgctgctag cgctatatgc gttgatgcaa | 2100 |
| tttctatgca ctcgtagtag tctgagaagg gtaacgccct ttacatggca aaggggtaca | 2160 |
| gttattgtgt actaaaatta aaaattgatt agggaggaaa acctcaaaat gaaaccaaca | 2220 |
| atggcaattt tagaaagaat cagtaaaaat tcacaagaaa atatagacga agtttttaca | 2280 |
| agactttatc gttatctttt acgtccagat atttattacg tggcgtatca aaatttatat | 2340 |
| tccaataaag gagcttccac aaaaggaata ttagatgata cagcggatgg ctttagtgaa | 2400 |
| gaaaaaataa aaaagattat tcaatcttta aaagacggaa cttactatcc tcaacctgta | 2460 |
| cgaagaatgt atattgcaaa aaagaattct aaaaagatga gacctttagg aattccaact | 2520 |
| ttcacagata aattgatcca agaagctgtg agaataattc ttgaatctat ctatgaaccg | 2580 |
| gtattcgaag atgtgtctca cggttttaga cctcaacgaa gctgtcacac agctttgaaa | 2640 |
| acaatcaaaa gagagtttgg cggcgcaaga tggtttgtgg agggagatat aaaaggctgc | 2700 |
| ttcgataata tagaccacgt tacactcatt ggactcatca atcttaaaat caaagatatg | 2760 |
| aaaatgagcc aattgattta taaatttcta aaagcaggtt atctggaaaa ctggcagtat | 2820 |
| cacaaaactt acagcggaac acctcaaggt ggaattctat ctcctctttt ggccaacatc | 2880 |
| tatcttcatg aattggataa gtttgtttta caactcaaaa tgaagtttga ccgagaaagt | 2940 |
| ccagaaagaa taacacctga atatcgggag ctccacaatg agataaaaag aatttctcac | 3000 |
| cgtctcaaga agttggaggg tgaagaaaaa gctaaagttc ttttagaata tcaagaaaaa | 3060 |
| cgtaaaagat tacccacact cccctgtacc tcacagacaa ataaagtatt gaaatacgtc | 3120 |
| cggtatgcgg acgacttcat tatctctgtt aaaggaagca aagaggactg tcaatggata | 3180 |
| aaagaacaat taaaactttt tattcataac aagctaaaaa tggaattgag tgaagaaaaa | 3240 |
| acactcatca cacatagcag tcaacccgct cgttttctgg gatatgatat acgagtaagg | 3300 |
| agatctggaa cgataaaacg atctggtaaa gtcaaaaaga gaacactcaa tgggagtgta | 3360 |
| gaactcctta ttcctcttca agacaaaatt cgtcaattta tttttgacaa gaaaatagct | 3420 |
| atccaaaaga aagatagctc atggtttcca gttcacagga aatatcttat tcgttcaaca | 3480 |
| gacttagaaa tcatcacaat ttataattct gaactccgcg ggatttgtaa ttactacggt | 3540 |
| ctagcaagta attttaacca gctcaattat tttgcttatc ttatggaata cagctgtcta | 3600 |
| aaaacgatag cctccaaaca taagggaaca ctttcaaaaa ccatttccat gtttaaagat | 3660 |
| ggaagtggtt cgtgggggat cccgtatgag ataaagcaag gtaagcagcg ccgttatttt | 3720 |
| gcaaatttta gtgaatgtaa atccccttat caatttacgg atgagataag tcaagctcct | 3780 |
| gtattgtatg gctatgcccg gaatactctt gaaaacaggt taaaagctaa atgttgtgaa | 3840 |
| ttatgtggga cgtctgatga aaatacttcc tatgaaattc accatgtcaa taaggtcaaa | 3900 |
| aatcttaaag gcaaagaaaa atgggaaatg gcaatgatag cgaaacaacg taaaactctt | 3960 |
| gttgtatgct ttcattgtca tcgtcacgtg attcataaac acaagtgaat gtcgagcacc | 4020 |
| cgttctcgga gcactgtccg accgctttgg ccgccgccca gtcctgctcg cttcgctact | 4080 |
| tggagccact atcgactacg cgatcatggc gaccacaccc gtcctgtgga tcgccaagcc | 4140 |
| gccgatggta gtgtggggtc tccccatgcg agagtaggga actgccaggc atcaaataaa | 4200 |
| acgaaaggct cagtcgaaag actgggcctt tcgttttatc tgttgtttgt cggtgaacgc | 4260 |
| tctcctgagt aggacaaatc cgccgggagc ggatttgaac gttgcgaagc aacggcccgg | 4320 |
| agggtggcgg gcaggacgcc cgccataaac tgccaggcat caaattaagc agaaggccat | 4380 |
| cctgacggat ggcctttttg cgtttctaca aactcttcct gtcgtcatat ctacaagcca | 4440 |
| tccccccaca gatacggtaa actagcctcg tttttgcatc aggaaagcag aacgccatga | 4500 |
| gcggcctcat ttcttattct gagttacaac agtccgcacc gctgccggta gctccttccg | 4560 |
| gtgggcgcgg ggcatgacta tcgtcgccgc acttatgact gtcttcttta tcatgcaact | 4620 |
| cgtaggacag gtgccggcag aggctaggtg gaggctcagt gatgataagt ctgcgatggt | 4680 |
| ggatgcatgt gtcatggtca tagctgtttc ctgtgtgaaa ttgttatccg ctcagagggc | 4740 |
| acaatcctat tccgcgctat ccgacaatct ccaagacatt aggtggagtt cagttcggcg | 4800 |
| agcggaaatg gcttacgaac ggggcggaga tttcctggaa gatgccagga agatacttaa | 4860 |
| cagggaagtg agagggccgc ggcaaagccg tttttccata ggctccgccc ccctgacaag | 4920 |
| catcacgaaa tctgacgctc aaatcagtgg tggcgaaacc cgacaggact ataaagatac | 4980 |
| caggcgtttc cccctggcgg ctccctcgtg cgctctcctg ttcctgcctt tcggtttacc | 5040 |
| ggtgtcattc cgctgttatg gccgcgtttg tctcattcca cgcctgacac tcagttccgg | 5100 |
| gtaggcagtt cgctccaagc tggactgtat gcacgaaccc cccgttcagt ccgaccgctg | 5160 |
| cgccttatcc ggtaactatc gtcttgagtc caacccggaa agacatgcaa aagcaccact | 5220 |
| ggcagcagcc actggtaatt gatttagagg agttagtctt gaagtcatgc gccggttaag | 5280 |
| gctaaactga aaggacaagt tttggtgact gcgctcctcc aagccagtta cctcggttca | 5340 |
| aagagttggt agctcagaga accttcgaaa aaccgccctg caaggcggtt ttttcgtttt | 5400 |
| cagagcaaga gattacgcgc agaccaaaac gatctcaaga agatcatctt attaagtctg | 5460 |
| acgctctatt caacaaagcc gccgtccatg ggtagggggc ttcaaatcgt ccccccatac | 5520 |
| gatataagtt gttactagtg cttggattct caccaataaa aaacgcccgg cggcaaccga | 5580 |
| gcgttctgaa caaatccaga tggagttctg aggtcattac tggatctatc aacaggagtc | 5640 |
| caagcgagct cgatatcaaa ttacgccccg ccctgccact catcgcagta ctgttgtaat | 5700 |
| tcattaagca ttctgccgac atggaagcca tcacaaacgg catgatgaac ctgaatcgcc | 5760 |
| agcggcatca gcaccttgtc gccttgcgta taatatttgc ccatggtgaa aacgggggcg | 5820 |
| aagaagttgt ccatattggc cacgtttaaa tcaaaactgg tgaaactcac ccagggattg | 5880 |
| gctgagacga aaaacatatt ctcaataaac cctttaggga aataggccag gttttcaccg | 5940 |
| taacacgcca catcttgcga atatatgtgt agaaactgcc ggaaatcgtc gtggtattca | 6000 |
| ctccagagcg atgaaaacgt ttcagtttgc tcatggaaaa cggtgtaaca agggtgaaca | 6060 |
| ctatcccata tcaccagctc accgtctttc attgccatac gaaattccgg atgagcattc | 6120 |
| atcaggcggg caagaatgtg aataaaggcc ggataaaact tgtgcttatt tttctttacg | 6180 |
| gtctttaaaa aggccgtaat atccagctga acggtctggt tataggtaca ttgagcaact | 6240 |
| gactgaaatg cctcaaaatg ttctttacga tgccattggg atatatcaac ggtggtatat | 6300 |
| ccagtgattt ttttctccat tttagcttcc ttagctcctg aaaatctcga taactcaaaa | 6360 |
| aatacgcccg gtagtgatct tatttcatta tggtgaaagt tggaacctct tacgtgccga | 6420 |
| tcaacgtctc attgatacct gaaacaaaac ccatcgtacg gccaaggaag tctccaataa | 6480 |
| ctgtgatcca ccacaagcgc cagggttttc ccagtcacga cgttgtaaaa cgacggccag | 6540 |
| tcatgcataa tccgcacgca tctggaataa ggaagtgcca ttccgcctga cct | 6593 |

<210> 17
<211> 364
<212> DNA
<213> Artificial sequence
<220>
<223> synthetic DNA
<400> 17

| | |
|---|---|
| ttcccctcta gaaaatctag aataattatc cttataggac gtcatggtgc gcccagatag | 60 |
| ggtgttaagt caagtagttt aaggtactac tctgtaagat aacacagaaa acagccaacc | 120 |
| taaccgaaaa gcgaaagctg atacgggaac agagcacggt tggaaagcga tgagttacct | 180 |
| aaagacaatc gggtacgact gagtcgcaat gttaatcaga tataaggtat aagttgtgtt | 240 |
| tactgaacgc aagtttctaa tttcgatttc ctatcgatag aggaaagtgt ctgaaacctc | 300 |
| tagtacaaag aaaggtaagt taaacatgac gacttatctg ttatcaccac atttgtacaa | 360 |
| tctg | 364 |
| <210> 18 | |
| <211> 24 | |
| <212> DNA | |
| <213> Artificial sequence | |
| <220> | |
| <223> primer | |
| <400> 18 | |
| aggggaattg tgagcggata acaa | 24 |
| <210> 19 | |
| <211> 18 | |
| <212> DNA | |
| <213> Artificial sequence | |
| <220> | |
| <223> primer | |
| <400> 19 | |
| ccagctggca attccggt | 18 |
| <210> 20 | |
| <211> 34 | |
| <212> DNA | |
| <213> Artificial sequence | |
| <220> | |
| <223> primer | |
| <400> 20 | |
| ttcccctcta gaaaatctag aataattatc ctta | 34 |
| <210> 21 | |
| <211> 34 | |
| <212> DNA | |
| <213> Artificial sequence | |
| <220> | |
| <223> primer | |
| <400> 21 | |
| cagattgtac aaatgtggtg ataacagata agtc | 34 |
| <210> 22 | |
| <211> 22 | |
| <212> DNA | |
| <213> Artificial sequence | |
| <220> | |
| <223> primer | |
| <400> 22 | |
| gatttggctg ccagttattt ag | 22 |
| <210> 23 | |
| <211> 21 | |
| <212> DNA | |
| <213> Artificial sequence | |
| <220> | |
| <223> primer | |
| <400> 23 | |
| gtcctttcct caaggttaat g | 21 |
| <210> 24 | |
| <211> 795 | |
| <212> DNA | |
| <213> Artificial sequence | |
| <220> | |
| <223> synthetic DNA | |
| <400> 24 | |
| atgaacctgc gcgaaaaata cggcgaatgg ggtatcattc tgggcgccac cgaaggcgtg | 60 |
| ggcaaagcct ttgcggaaaa aattgcaagt gaaggcatga gcgtggtgct ggtgggccgc | 120 |
| cgcgaagaaa aactgcagga actgggcaaa agcattagcg aaacctatgg cgttgatcat | 180 |
| atggtgattc gcgccgattt tgcgcagagc gattgcaccg ataaaatttt tgaagcgacc | 240 |
| aaagatctgg atatgggctt tatgagttac gtggcatgct ttcatacctt tggcaaactg | 300 |
| caggataccc cgtgggaaaa acatgaacag atgattaacg tgaatgttat gacctttctg | 360 |
| aaatgcttct atcattatat gggcattttt gccaaacagg atcgcggcgc ggtaattaat | 420 |
| gtgagcagcc tgaccgcgat tagtagcagc ccgtataacg cgcagtatgg cgcgggcaaa | 480 |
| tcgtacatta aaaaactgac ggaagccgtg goggccgaat gcgaaagcac caatgtggac | 540 |
| gtggaagtca ttaccctggg caccgtgatt accccgagcc tgctgagcaa cctgccgggc | 600 |
| ggcccggccg gcgaagccat gatgaaaacc gcgatgacgc cggaagcctg cgtggaagaa | 660 |
| gcgtttgaca acctgggcaa aagcctgagc gtgattgcgg gcgaacacaa caaagccaat | 720 |
| gttcataact ggcaggcgaa caaaaccgat gatgaatata ttcgctatat gggcagcttt | 780 |
| tatagcaata actaa | 795 |
| <210> 25 | |
| <211> 72 | |
| <212> DNA | |
| <213> Artificial sequence | |
| <220> | |
| <223> primer | |
| <400> 25 | |
| caattcccct ctagaaataa ttttgtttaa ctttaagaag gagatatacc atgaacctgc | 60 |
| gcgaaaaata cg | 72 |
| <210> 26 | |
| <211> 75 | |
| <212> DNA | |
| <213> Artificial sequence | |
| <220> | |
| <223> primer | |
| <400> 26 | |
| ctcaagaccc gtttagaggc cccaaggggt tatgctagtt attgctcagc ttagttattg | 60 |
| ctataaaagc tgccc | 75 |
| <210> 27 | |
| <211> 23 | |
| <212> DNA | |
| <213> Artificial sequence | |
| <220> | |
| <223> primer | |
| <400> 27 | |
| ccaaaatccc ttaacgtgagttt | 23 |
| <210> 28 | |
| <211> 30 | |
| <212> DNA | |
| <213> Artificial sequence | |
| <220> | |
| <223> primer | |
| <400> 28 | |
| gaattaattc atgagcggat acatatttga | 30 |
| <210> 29 | |
| <211> 966 | |
| <212> DNA | |
| <213> Artificial sequence | |
| <220> | |
| <223> synthetic DNA | |
| <400> 29 | |
| cgcggaaccc ctatttgttt atttttctaa atacattcaa atatgtatcc gctcatgaga | 60 |
| caataaccct gataaatgct tcaataatat tgaaaaagga agagtatgag tattcaacat | 120 |
| ttccgtgtcg cccttattcc cttttttgcg gcattttgcc ttcctgtttt tgctcaccca | 180 |
| gaaacgctgg tgaaagtaaa agatgctgaa gatcagttgg gtgcacgagt gggttacatc | 240 |
| gaactggatc tcaacagcgg taagatcctt gagagttttc gccccgaaga acgttttcca | 300 |
| atgatgagca cttttaaagt tctgctatgt ggcgcggtat tatcccgtat tgacgccggg | 360 |
| caagagcaac tcggtcgccg catacactat tctcagaatg acttggttga gtactcacca | 420 |
| gtcacagaaa agcatcttac ggatggcatg acagtaagag aattatgcag tgctgccata | 480 |
| accatgagtg ataacactgc ggccaactta cttctgacaa cgatcggagg accgaaggag | 540 |
| ctaaccgctt ttttgcacaa catgggggat catgtaactc gccttgatcg ttgggaaccg | 600 |
| gagctgaatg aagccatacc aaacgacgag cgtgacacca cgatgcctgt agcaatggca | 660 |
| acaacgttgc gcaaactatt aactggcgaa ctacttactc tagcttcccg gcaacaatta | 720 |
| atagactgga tggaggcgga taaagttgca ggaccacttc tgcgctcggc ccttccggct | 780 |
| ggctggttta ttgctgataa atctggagcc ggtgagcgtg ggtctcgcgg tatcattgca | 840 |
| gcactggggc cagatggtaa gccctcccgt atcgtagtta tctacacgac ggggagtcag | 900 |
| gcaactatgg atgaacgaaa tagacagatc gctgagatag gtgcctcact gattaagcat | 960 |
| tggtaa | 966 |
| <210> 30 | |
| <211> 75 | |
| <212> DNA | |
| <213> Artificial sequence | |
| <220> | |
| <223> primer | |
| <400> 30 | |
| tacggggtct gacgctcagt ggaacgaaaa ctcacgttaa gggattttgg cgcggaaccc | 60 |
| ctatttgttt atttt | 75 |
| <210> 31 | |
| <211> 75 | |
| <212> DNA | |
| <213> Artificial sequence | |
| <220> | |
| <223> primer | |
| <400> 31 | |
| gtttattttt ctaaatacat tcaaatatgt atccgctcat gaattaattc ttaccaatgc | 60 |
| ttaatcagtg aggca | 75 |
| * * * * * * * * | |

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A compound selected from ursodeoxycholic acid of formula I: 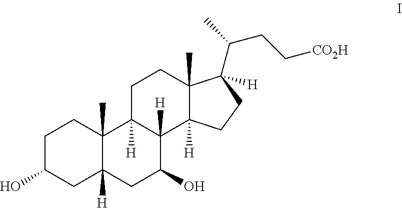
and its pharmaceutically acceptable salts comprising a δ¹³C value corresponding to a plant derived molecule, and an impurity profile characterized by:
a) less than 5%, 3%, 1%, 0.5%, 0.1%, 0.05%, 0.03%, or 0.01% of any 3β-hydroxysteroids;
b) less than 5%, 3%, 1%, 0.5%, 0.1%, 0.05%, 0.03%, or 0.01% of any 5α-steroids; and
c) less than 5%, 3%, 1%, 0.5%, 0.1%, 0.05%, 0.03%, or 0.01% of any 7α-hydroxysteroids.

2) (canceled)

3) (canceled)

4) The compound of claim 1 further comprising:
a) less than 5%, 3%, 1%, 0.5%, 0.1%, 0.05%, 0.03%, or 0.01% of LCA;
b) less than 5%, 3%, 1%, 0.5%, 0.1%, 0.05%, 0.03%, or 0.01% of any 3-keto, 7-hydroxysteroids;
c) less than 5%, 3%, 1%, 0.5%, 0.1%, 0.05%, 0.03%, or 0.01% of any 3-hydroxy, 7-ketosteroids; and/or
d) less than 5%, 3%, 1%, 0.5%, 0.1%, 0.05%, 0.03%, or 0.01% of DKCA.

5) (canceled)

6) (canceled)

7) (canceled)

8) (canceled)

9) The compound of claim 1 comprising an impurity profile characterized by:
a) less than 0.10% of any 3β-hydroxysteroids;
b) less than 0.5% of any 5α-steroids; and
c) less than 0.10% of any 7α-hydroxysteroids.

10) (canceled)

11) A compound selected from ursodeoxycholic acid of formula I: 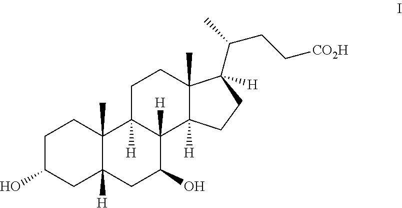
and its pharmaceutically acceptable salts comprising a δ¹³C value corresponding to a plant derived molecule and an impurity profile characterized by:
a) less than 0.05%, 0.03%, or 0.01% of any 3β-hydroxysteroids; or
b) less than 0.05%, 0.03%, or 0.01% of any 5α-steroids; or
c) less than 0.05%, 0.03%, or 0.01% of any 7α-steroids.

12) The compound of claim 11 comprising an impurity profile characterized by:
a) less than 0.01% of any 3β-hydroxysteroids; or
b) less than 0.01% of any 5α-steroids; or
c) less than 0.01% of any 7α-hydroxysteroids.

13) The compound of claim 11 comprising an impurity profile characterized by
a) less than 0.05%, 0.03%, or 0.01% of any 3β-hydroxysteroids;
b) less than 0.05%, 0.03%, or 0.01% of any 5α-steroids; and
c) less than 0.05%, 0.03%, or 0.01% of any 7α-hydroxysteroids.

14) The compound of claim 11 comprising an impurity profile characterized by:
a) less than 0.01% of any 3β-hydroxysteroids;
b) less than 0.01% of any 5α-steroids; and
c) less than 0.01% of any 7α-hydroxysteroids.

15) (canceled)

16) (canceled)

17) (canceled)

18) (canceled)

19) (canceled)

20) A method of making a UDCA pharmaceutical dosage form comprising admixing the compound of claim 1 with one or more pharmaceutically acceptable excipients to form an admixture and processing the admixture into a finished dosage form, preferably by compressing the admixture into a tablet or filling the admixture into a capsule or sachet.

21) A method of producing the compound of claim 1 that goes through a DKCA intermediate, comprising:
a) contacting the DKCA with a 3α-hydroxysteroid dehydrogenase to stereo-selectively reduce the DKCA to a 3α hydroxy intermediate, and contacting the 3α hydroxy intermediate with a 7β-hydroxysteroid dehydrogenase to stereo-selectively reduce the 3α hydroxy intermediate to UDCA; or
b) contacting the DKCA with a 7β-hydroxysteroid dehydrogenase to stereo-selectively reduce the DKCA to a 7β hydroxy intermediate, and contacting the 7β hydroxy intermediate with a 3α-hydroxysteroid dehydrogenase to stereo-selectively reduce the 7β hydroxy intermediate to UDCA; or
c) simultaneously contacting the DKCA with a 3α-hydroxysteroid dehydrogenase and a 7β-hydroxysteroid dehydrogenase to stereo-selectively reduce the DKCA to UDCA.

22) (canceled)

23) (canceled)

24) (canceled)

25) (canceled)

26) (canceled)

27) (canceled)

28) (canceled)

29) (canceled)

30) A tert-butylamine salt of 3,7-DKCA having crystalline form Pattern 9-A defined by:
a) an XRPD pattern comprising at least one, two, or three peaks in terms of 20, selected from the group consisting of 4.83, 8.77, 13.35 15.56, 16.03, 20.54, 22.05, 23.53, 24.75, 29.93, 30.40, and 31.970±0.2°, or
b) an XRPD pattern substantially as depicted in FIG. 3.

31) (canceled)

32) A diisopropylamine salt of 3,7-DKCA having crystalline form Pattern 10-A defined by
a) an XRPD pattern comprising at least one, two, or three peaks in terms of 20, selected from the group consisting of 5.85, 6.29, 9.05, 12.58, 14.17, 16.09, 18.13, 18.47, 18.89, 20.49, 21.48, 24.75, 25.27, 28.65, 30.21, 31.82, 34.78, and 37.44°±0.2°, or
b) has an XRPD pattern substantially as depicted in FIG. 5.

33) (canceled)

34) (canceled)

35) The compound of claim 1 comprising an impurity profile characterized by:
a) less than 1% of any 3β-hydroxysteroids;
b) less than 3% of any 5α-steroids; and
c) less than 1% of any 7α-hydroxysteroids.

36) The compound of claim 1 comprising an impurity profile characterized by:
a) less than 0.5% of any 3β-hydroxysteroids;
b) less than 1% of any 5α-steroids; and
c) less than 0.5% of any 7α-hydroxysteroids.

37) The compound of claim 1 comprising less than −15‰, −17.5‰, −20‰, −22.5‰, or −25‰ δ13C relative to VPDB.

38) The compound of claim 1 in an isolated state.

39) The compound of claim 11 comprising less than −15‰, −17.5‰, −20‰, −22.5‰, or −25‰ δ13C relative to VPDB.

40) The compound of claim 11 in an isolated state.

41) The compound of claim 30 in an isolated state.
